SEQUENCE LISTING (4 sequences)

SEQ ID NO: 1 - protein, 679 aa, Homo sapiens
  MISC_FEATURE (1)..(679): Homo sapiens transferrin
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 2 - protein, 691 aa, Homo sapiens
  MISC_FEATURE (1)..(691): Homo sapiens lactoferrin
GRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWAGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQAGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGADCPDKFCLFQSETKNLLFNDNTECLARLHGKTTYEKYLGPQYVAGITNLKKCSTSPLLEACEFLRK

SEQ ID NO: 3 - protein, 738 aa, Homo sapiens
  MISC_FEATURE (1)..(738): Homo sapiens lactoferrin
MRGPSGALWLLLALRTVLGGMEVRWCATSDPEQHKCGNMSEAFREAGIQPSLLCVRGTSADHCVQLIAAQEADAITLDGGAIYEAGKEHGLKPVVGEVYDQEVGTSYYAVAVVRRSSHVTIDTLKGVKSCHTGINRTVGWNVPVGYLVESGRLSVMGCDVLKAVSDYFGGSCVPGAGETSYSESLCRLCRGDSSGEGVCDKSPLERYYDYSGAFRCLAEGAGDVAFVKHSTVLENTDGKTLPSWGQALLSQDFELLCRDGSRADVTEWRQCHLARVPAHAVVVRADTDGGLIFRLLNEGQRLFSHEGSSFQMFSSEAYGQKDLLFKDSTSELVPIATQTYEAWLGHEYLHAMKGLLCDPNRLPPYLRWCVLSTPEIQKCGDMAVAFRRQRLKPEIQCVSAKSPQHCMERIQAEQVDAVTLSGEDIYTAGKTYGLVPAAGEHYAPEDSSNSYYVVAVVRRDSSHAFTLDELRGKRSCHAGFGSPAGWDVPVGALIQRGFIRPKDCDVLTAVSEFFNASCVPVNNPKNYPSSLCALCVGDEQGRNKCVGNSQERYYGYRGAFRCLVENAGDVAFVRHTTVFDNTNGHNSEPWAAELRSEDYELLCPNGARAEVSQFAACNLAQIPPHAVMVRPDTNIFTVYGLLDKAQDLFGDDHNKNGFKMFDSSNYHGQDLLFKDATVRAVPVGEKTTYRGWLGLDYVAALEGMSSQQCSGAAAPAPGAPLLPLLLPALAARLLPPAL

SEQ ID NO: 4 - protein, 679 aa, Artificial sequence
  Artificial variant of Homo sapiens transferrin
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKADNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVADCSGNFCLFRSETKDLLFRDDTVCLAKLHDRNTYEKYLGEEYVKAVGNLRKCSTSSLLEACTFRRP

(19) United States
(12) Patent Application Publication
(10) Pub. No.: US 2011/0294988 A1
(43) Pub. Date: Dec. 1, 2011

(54) Purification Process

(75) Inventors: Lee Edward Blackwell, Nottinghamshire (GB); Jason Cameron, Nottinghamshire (GB); Phillip Harvey Morton, Nottinghamshire (GB)

(73) Assignee: Novozymes Bio Pharma DK A/S, Bagsvaerd (DK)

(21) Appl. No.: 13/148,260

(22) PCT Filed: Feb. 5, 2010
(86) PCT No.: PCT/EP2010/051453

§ 371 Date: Aug. 5, 2011

(30) Foreign Application Priority Data
Feb. 6, 2009 (EP) 09152332.4

(51) Int. Cl. C07K 14/79; C07K 1/18

(52) U.S. Cl. 530/394

(57) ABSTRACT

A process for purifying a transferrin solution and the product of the purification process. A process for producing transferrin and the product of the production process. Use of the transferrin in cell culture, as a pharmaceutical ingredient, as a pharmaceutical product and in a cell culture medium.

### REFERENCE TO SEQUENCE LISTING

This application contains a Sequence Listing in computer readable form. The computer readable form is incorporated herein by reference.

### FIELD OF THE INVENTION

The invention relates to a process for purifying a transferrin solution. The invention also relates to the product of the purification process. The term ‘transferrin’ refers to both transferrin and transferrin-like proteins.

### BACKGROUND TO THE INVENTION

The listing or discussion of a prior-published document in this specification should not necessarily be taken as an acknowledgement that the document is part of the state of the art or is common general knowledge.

Transferrin is a protein that is known to regulate the bioavailability of iron to the cell, supporting key metabolic processes such as DNA synthesis and oxygen transport. Human serum transferrin (HST) is the major iron-binding protein in normal human plasma, and is present at about 2-4 g/l (van Campenhout et al, 2003, Free Radic. Res., 37, 1069-1077). Serum transferrin is currently derived from either bovine or human sources and is a common supplement used in commercial scale mammalian cell culture. With increasing pressure from regulatory authorities to reduce animal-derived components in the manufacture of biologics, recombinant transferrin is desirable for the serum-free, large scale mammalian cell culture industry.

More specifically, human plasma transferrin is a monomeric glycoprotein of 678 amino acids with a relative molecular mass of about 80 kDa. Each molecule of transferrin can bind strongly one or two ferric ions.

Purification of transferrin from plasma by chromatography has been described, for example in Rivet et al (1992) Journal of Chromatography, 576: 71-77, U.S. Pat. No. 6,251,860; U.S. Pat. No. 5,744,586; and U.S. Pat. No. 5,041,537. Purification of recombinant transferrin from yeast has been described in PCT/EP2008/060482, and U.S. Pat. No. 5,986,067. Transferrin may also be produced in other hosts, such as in plants

Irrespective of the source of transferrin, be it animal, human or recombinant, it is desirable to recover maximal amounts of transferrin during purification of the transferrin from the source. However, current methods of purification of transferrin do not result in a sufficiently high yield of purified transferrin. Therefore, what is required is an improved purification process for transferrin.

### SUMMARY OF THE INVENTION

The invention provides a chromatographic purification process which results in an increased yield of transferrin. Two steps of the purification process have been found to be particularly beneficial. These two steps may be used independently or together.

Accordingly, the invention provides an improved process for the purification of transferrin from a relatively impure solution of transferrin. The process may include one or both of an anionic chromatography step and a cationic chromatography step.

The invention also provides transferrin purified by the purification method and uses of the transferrin purified by the purification method.

### BRIEF DESCRIPTION OF DRAWINGS

FIG. 1 shows A254 nm trace of Capto™ Q chromatograph with (A) zero and (B) 824 mol.mole⁻¹ (5.4 mM) borate present in the transferrin solution which is loaded onto the chromatography column.

FIG. 2 is a graphical representation, overlay plot, of the SP-FF low pH, high salt wash 2 design space for a host cell protein clearance of at least 275-fold and a recovery of at least 60%.

### DETAILED DESCRIPTION OF THE INVENTION

A first aspect of the invention provides a process for purifying a transferrin solution (the transferrin solution may be considered to be a ‘starting material’), the process comprising:
a) adding tetraborate to a relatively impure solution of transferrin to provide a solution of transferrin and tetraborate;
b) applying the solution of transferrin and tetraborate to an anionic chromatographic material for which the transferrin has no specific binding activity such that the transferrin binds to the material;
c) washing the chromatographic material to which the transferrin is bound;
d) optionally further washing the chromatographic material to which the transferrin is bound; and
e) optionally, recovering the transferrin from the anionic chromatographic material.

Preferably the wash of step (c) is carried out using a wash solution having a lower concentration than the wash solution used in the wash of step (d). For example, ‘lower’ may mean at least 5, 10, 25, 30 or 50% lower and/or by at least 5, 10, 20, 30, 40, or 50 mM or by at least 2-, 3-, 4-, 5- or 10-fold.

More specifically, the process for purifying a transferrin solution may comprise the steps of:
a) adding tetraborate to a relatively impure solution of transferrin to provide a solution of transferrin and tetraborate at a ratio of at least 1 mole tetraborate to 1 mole transferrin, for example at least 20 mole tetraborate to 1 mole transferrin;
b) applying the solution of transferrin and tetraborate to an anionic chromatographic material for which the transferrin has no specific binding affinity such that the transferrin-binds to the material;
c) washing the chromatographic material with a tetraborate solution of from 0.1 mM to 30 mM, for example 0.1 mM to 20 mM;
d) subsequently washing the chromatographic material with a tetraborate solution of from 5 mM to 60 mM, for example 30 to 60 mM or at least 30 mM; and
e) optionally, recovering the transferrin from the anionic chromatographic material.

The term ‘no specific binding activity’ means that, preferably, the transferrin does not bind to the chromatographic material in an antibody-antigen binding manner.

A surprising advantage of adding tetraborate to the transferrin solution prior to loading the transferrin on to the anionic chromatography material is that this increases the load capacity of the material for transferrin. The load capacity may be increased by 2-fold, 3-fold or more. Thus more transferrin may be loaded on a given amount of matrix, and/or the amount of transferrin lost in the flow-through may be reduced. Preferably, tetraborate is added at a ratio from 1 mole tetraborate to 1 mole transferrin to 1000 mole tetraborate to 1 mole transferrin, for example at, from or up to at or about 1, 10, 15, 20, 30, 50, 100, 200, 300, 400, 450, 500, 600, 700, 800, 900 or 1000 mol.mol⁻¹. A preferred ratio is from 20 to 1000 mole tetraborate to 1 mole transferrin. The advantage of adding tetraborate to transferrin prior to loading the transferrin onto the anionic chromatographic material may be exploited alone or in combination with the wash step(s) of the purification process disclosed above. For example, tetraborate may be used to increase the transferrin-binding capacity of an anionic exchange matrix independently of or in combination with a purification process that uses that matrix.

The tetraborate solution used in the washing of step (c) may be at a concentration of from at or about 0.1 mM to at or about 30 mM. For example, the tetraborate solution used in (c) may be at a concentration from at or about 0.1 mM, 0.5 mM, 1 mM, 2 mM, 4 mM, 6 mM, 8 mM, 10 mM, 15 mM, 20 mM or 25 mM to at or about 0.5 mM, 1 mM, 2 mM, 4 mM, 6 mM, 8 mM, 10 mM, 15 mM, 20 mM, 25 mM or 30 mM. More particularly the tetraborate solution of (c) may be at a concentration from at or about 2.5 mM to at or about 7.5 mM, most particularly at or about 5 mM.

The tetraborate solution used in the washing of step (d) may be at a concentration from at or about 5 mM to at or about 60 mM. For example, the tetraborate solution used in (d) may be at a concentration from at or about 5 mM, 10 mM, 15 mM, 20 mM, 25 mM, 30 mM, 35 mM, 40 mM, 45 mM, 50 mM or 55 mM to at or about 10 mM, 15 mM, 20 mM, 25 mM, 30 mM, 35 mM, 40 mM, 45 mM, 50 mM, 55 mM or 60 mM. More particularly the tetraborate solution of (d) may be at a concentration from at or about 20 mM to at or about 40 mM, most particularly at or about 30 mM.

A surprising advantage of carrying out a low concentration ‘pre-wash’, such as the wash of step (c), prior to a higher concentration wash, such as the wash of step (d), is that this regime reduces the loss of transferrin during the higher concentration wash. Preferably, ‘concentration’ is ‘tetraborate concentration’.

The tetraborate may be a tetraborate of a Group I metal, such as potassium tetraborate or sodium tetraborate. A preferred tetraborate is potassium tetraborate.

The transferrin may be recovered from the anionic chromatographic material by elution, for example by a change in pH and/or an increase in conductivity. The elution solution may have a pH of less than or equal to pH 5.2 and/or a conductivity of greater than or equal to 2.5 mS.cm⁻¹. An alternative elution liquid comprises 110 mM potassium tetraborate or sodium tetraborate.

Preferably the anionic chromatographic material comprises a functional ligand which is a strong anion exchanger or a weak anion exchanger. A functional ligand may be an ion. Examples of strong anion exchangers include quaternary amine groups (“Q”), i.e. —N⁺ (CH₃)₃ such as Q Sepharose Fast Flow (Q-FF) and Capto Q (both available from GE Healthcare, Little Chalfont, UK), UNOsphere™ Q (Bio-Rad, Hemel Hempstead, UK), Toyopearl® SuperQ (TosoH Corporation, Tokyo, Japan), Q Ceramic HyperD® (Pall, Portsmouth, UK) and POROS® 50 HQ (Applied Biosystems Inc., Foster City, Calif., USA). Examples of weak anion exchangers include diethylaminoalkyl groups such as diethylaminoethyl groups (“DEAE”, also referred to as “DE”) i.e. —N⁺H(CH₂CH₃)₂ such as DEAE Sepharose Fast Flow (DE-FF; available from GE Healthcare). Further strong and weak anion exchangers are described in ‘Liquid column chromatography: a survey of modern techniques and applications’ (1975, pages 344-351, Editors: Zden\check{e}k Deyl, Karel Macek, Jaroslav Janák, Publisher: Elsevier) and in ‘Protein liquid chromatography’ (2000, pages 18-21, Editor: Michael Kastener, Publisher: Elsevier) both of which are hereby incorporated by reference.

Weak anion exchangers may benefit from the use of wash buffers for steps (c) and (d) which are of a lower concentration than those used for strong anion exchangers.

Preferably the transferrin solution is loaded onto the anionic chromatography material at from about 0.01 mg transferrin per mL chromatography matrix to about 100 mg.mL⁻¹., for example about from at or about 0.01, 0.1, 1, 10, 20, 30, 40, 50, 60, 70, 80 or 90 mg.mL⁻¹ to at or about 0.1, 1, 10, 20, 30, 40, 50, 60, 70, 80, 90 or 100 mg.mL⁻¹. Preferred loading parameters are from at or about 10 to at or about 100 mg.mL⁻¹. A particularly preferred loading parameter is at or about 30 mg.mL⁻¹.

Preferably the transferrin solution which is loaded onto the anionic chromatography matrix has a pH of from about pH 8 to 10, such as from about pH 8.0 to 10.0 more preferably at, to or from pH 9.0 to 9.4.

Prior to loading the transferrin-containing material onto the anionic chromatographic material, the anionic chromatographic material may be equilibrated for example with one or more strengths of tetraborate solution, for example 110 mM potassium tetraborate and/or 30 mM potassium tetraborate. Potassium tetraborate may be partially or fully replaced by a different tetraborate such as a Group I metal tetraborate, such as sodium tetraborate.

A second aspect of the invention provides a process for purifying a transferrin solution (the transferrin solution may be considered to be a ‘starting material’), the process comprising a cationic chromatographic step comprising the steps of:
a) applying a relatively impure solution of a transferrin to a cationic chromatographic material for which the transferrin has no specific binding affinity such that the transferrin binds to the material;
b) washing the chromatographic material with a wash buffer having a pH of from about 4 to about 5 and a salt concentration of 200 mM or less;
c) subsequently washing the chromatographic material with a wash buffer having a pH from about 4 to about 5 and a salt concentration of from about 50 mM to about 5 M; and
d) optionally, recovering the transferrin from the cationic chromatographic material.

Preferably the pH of the wash buffer of step (b) of the cationic chromatographic step is from at or about 4.0, 4.1, 4.2, 4.3, 4.4, 4.5, 4.6, 4.7, 4.8 or 4.9 to at or about 4.1, 4.2, 4.3, 4.4, 4.5, 4.6, 4.7, 4.8, 4.9 or 5.0, most preferably at or about pH 4.5.

Preferably the wash buffer of step (b) of the cationic chromatographic step has a total salt concentration of from about 10 mM to about 200 mM, more preferably at about 50 mM. More preferably the salt which provides the buffering activity of the wash buffer of step (b) of the cationic chromatographic step is present at a concentration of from about 10 mM to about 200 mM, more preferably at about 50 mM. Preferably the wash buffer of step (b) is substantially free of salt, other than the salt providing the buffering effect (i.e. the ‘buffering salt’) and most preferably is free of salt other than the buffering salt. The term ‘substantially free’ means that the level of salt, other than buffering salt, is undetectable and or is sufficiently low as to have no effect on the wash buffer relative to a wash buffer that is completely free of salt other than the buffering salt. For example, it is preferred that the wash buffer of step (b) is substantially free of sodium chloride.

It is preferred that the pH of the wash buffer of step (c) of the cationic chromatographic step is at or about 4.0, 4.1, 4.2, 4.3, 4.4, 4.5, 4.6, 4.7, 4.8 or 4.9 to at or about 4.1, 4.2, 4.3, 4.4, 4.5, 4.6, 4.7, 4.8, 4.9 or 5.0, most preferably at or about pH 4.5.

Preferably the wash buffer of step (c) of the cationic chromatographic step has a salt concentration of from about 50 mM to about 5 M, for example from at or about 50 mM, 100 mM, 150 mM, 200 mM, 250 mM, 500 mM, 1 M, 1.25 M, 1.5 M, 1.75 M, 2 M, 2.5 M, 3 M, 3.5 M, 4 M or 4.5 M to at or about 100 mM, 150 mM, 200 mM, 250 mM, 500 mM, 1 M, 1.25 M, 1.5 M, 1.75 M, 2 M, 2.5 M, 3 M, 3.5 M, 4 M, 4.5 M or 5 M, more preferably from at or about 250 mM to at or about 2 M, most preferably at or about 2 M. It is preferred that the wash buffer of step (c) has a higher salt concentration that the wash buffer of step (b). Example of ‘higher’ include by greater than or equal to 5, 10, 20, 30, 40, 50, 100, 150 mM.

Preferred wash buffers of step (c) of the cationic chromatographic step provide a recovert' of at least 60, 70, 80, 90, 95 or 100% and/or a host cell protein (HCP) clearance (e.g. yeast antigen clearance) of at least 200, 225, 250, 275, 300, 325, 350, 375 or 400-fold. Particularly preferred wash buffers provide a recovery of at least 60, 70, 80, 90, 95 or 100% and a HCP (e.g. YA) clearance of at least 200, 225, 250, 275, 300, 325, 350, 375 or 400 and are defined by, that is satisfies, one or more (preferably both) of the following equations:

Recovery=−940.50069+(441.61018×p)+(0.043309×s)−(47.57735×p²)−(0.010263×p×s)

HCP(e.g. YA)clearance=16388.65744−(10512.30681×p)−(1.70101×s)+(2257.17601×p²)+(0.78628×p×s)−(159.06573×p³)−(0.090182×p²×s)

In the above equations, p=pH and s=salt concentration in mM and recovery is % recovery (for example weight/weight).

For example, the wash buffer may provide a recovery of at least 60% and a HCP (e.g. YA) clearance of at least 275-fold. Parameters of a wash buffer providing a recovery of at least 60% and a HCP (e.g. YA) clearance of at least 275-fold may be defined by the following equations:

Recovery(%):60=−940.50069+(441.61018×p)+(0.043309×s)−(47.57735×p²)−(0.010263×p×s)

HCP(e.g. YA)clearance(fold):275=16388.65744−(10512.30681×p)−(1.70101×s)+(2257.17601×p²)+(0.78628×p×s)−(159.06573×p³)−(0.090182p²×s)

In the above equations, p pH and s=salt concentration in mM.

Recovery (%) may be weight/weight. The term ‘host cell protein’ is well known to the skilled person. Host cell proteins include proteins native to and produced by a host cell which may be present as a contaminant in a protein, such as a heterologous protein, produced by the host cell. It is desirable to reduce, or eliminate, the amount of host cell protein present in a product produced from that host cell. For a yeast host cell, the host cell proteins may be known as ‘yeast antigens’.

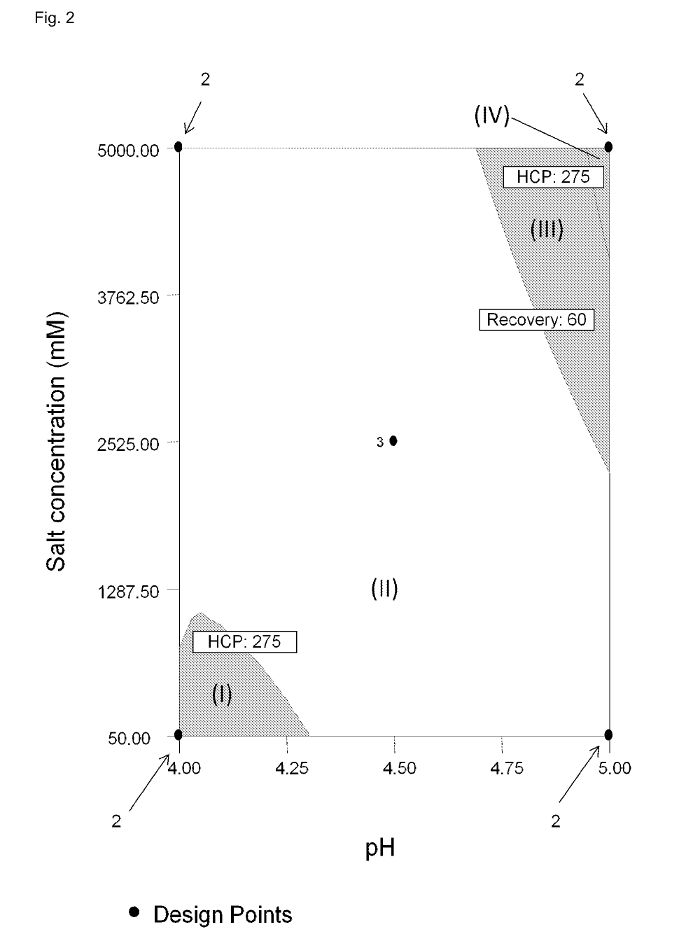

The pH, salt concentration, HCP (e.g. YA) clearance and recovery may be plotted on a graph, e.g. a an overlay plot. FIG. 2 shows an example of an overlay plot. Such a graph gives a view of the ‘design space’. The design space represents the combinations of pHs and salt concentrations at which the desired recovery (%) and HCP (e.g. YA) clearance (fold) is achieved. A design space can be generated (defined') for any combination of desired recovery and HCP(YA) clearance.

The design space is the most accurate definition of the combinations of pHs and salt concentrations which provide the desired recovery and HCP (e.g. YA) clearance. However, the combinations may also be approximated by describing at least a part, and preferably a significant part, of the design space by one or more rectangular areas. For example, the design space defined by a recovery of 60% or more and a HCP (e.g. YA) clearance of 275-fold or more may be approximated by one or more of (i) pH 4.3-5.0, 50-1100 mM salt, (ii) pH 4.0-4.7, 1100-5000 mM salt, (iii) pH 4.7-5.0, 1100-2270 mM salt (iv) pH 4.7-4.8, 2270-3900 mM salt, (v) pH 4.8-4.9, 2270-2990 mM salt and (vi) pH 4.7-4.75, 3900-4345 mM salt. Preferably the design is approximated by (i) to (iv), more preferably by (i) to (v) and most preferably by (i) to (vi). Suitable salts include monovalent and divalent metal and ammonium salts. One preferred salt for the cationic chromatographic step is NaCl.

The wash buffer of step (c) may or may not comprise two or more salts. For example, the wash buffer of step (c) may or may not comprise sodium acetate and NaCl. Sodium acetate may be present at or about 27 mM.

A surprising advantage of low pH/high salt wash, such as those described above, is that this improves HCP clearance, e.g. yeast antigen clearance, across the cationic matrix without significantly lowering the recovery of transferrin from the matrix. This is in direct contrast to the expectation that use of such a high salt wash would result in an undesirably high loss of transferrin from the matrix.

The transferrin may be recovered from the cationic chromatographic material by elution, for example with a change of pH and/or conductivity and/or the addition of a specific eluent. The elution solution may comprise sodium phosphate and or sodium chloride. For example sodium phosphate may be present at or about 50 mM. Sodium chloride may be present at or about 150 mM. The pH of the elution solution may be at or about pH 6, for example at or about pH 6.0.

The cationic chromatographic step may comprise a further, for example a third wash, prior to recovery of the transferrin. Preferably the further wash step is carried out after the wash step of step (c). Preferably, the further wash step is carried out immediately after the wash step of step (c), e.g. with no intervening step. The further wash may be carried out using a wash buffer of 50 mM sodium acetate pH 5.0 to 5.2 (preferably pH 5.1) preferably with a conductivity of from 2.5 to 3.1 mS.cm⁻¹.

Preferably the transferrin solution is loaded onto the cationic chromatography material at up to 100 mg transferrin per mL chromatographic material, for example from at or about 10, 20, 30, 40, 50, 60, 70, 80 or 90 mg.mL⁻¹ to at or about 20, 30, 40, 50, 60, 70, 80, 90 or 100 mg.mL⁻¹ more preferably at from about 30 to about 50 mg.mL⁻¹.

Preferably the cationic chromatographic material comprises a functional ligand which is a strong cation exchanger. Examples of strong cation exchangers include a sulphonic group, i.e.—SO₃⁻ e.g. SP Sepharose Fast Flow (SP-FF) and S-Spheradex; (both available from GE Healthcare), SP-Spherosil, SE-Cellulose A particularly preferred cationic chromatographic material is SP Sepharose Fast Flow. The cationic chromatographic material may comprise a functional ligand which is a weak cation exchanger, such materials include, CM-Sepharose FF or CM-Cellulose. Further strong and weak cation exchangers are described in ‘Liquid column chromatography: a survey of modern techniques and applications’ (1975, pages 344-351, Editors: Zden\check{e}k Deyl, Karel Macek, Jaroslav Janák, Publisher: Elsevier) and in ‘Protein liquid chromatography’ (2000, pages 18-21, Editor: Michael Kastener, Publisher: Elsevier).

Prior to loading (for example, step (a)), the cationic chromatographic material may be equilibrated for example with 50 mM sodium acetate (from at or about pH 4 to pH 6, such as at or about pH 4.0 to pH 6.0, for example from at or about pH 5.0 to at or about pH 5.2, preferably at or about pH 5.1 and with a conductivity of from at or about 2.5 mS.cm⁻¹ to at or about 3.1 mS.cm⁻¹).

Following recovery of the transferrin, the cationic and/or anionic chromatographic material may be cleaned prior to storage and/or re-use of the material. Techniques for cleaning chromatographic materials are known in the art.

A third aspect of the invention provides a process for purifying a transferrin solution, the process comprising a cationic step according to the second aspect of the invention and an anionic chromatographic step according to the first aspect of the invention. Preferably, the cationic step is followed by the anionic step. Alternatively, the cationic step may follow the anionic step. Preferably, the cationic is immediately followed by the anionic step, e.g. with no intervening step. Optionally, other steps may be carried out in between the cationic and anionic steps. The eluate of the cationic step may or may not be diluted prior to being subjected to the anionic step.

Preferably the transferrin solution which is subjected to the purification of the invention is substantially, or completely, free of particles such as cells and cell debris. ‘Substantially free’ means that any particles present in the solution do not have a significant adverse effect on the purification process. Preferably, any particles present do not reduce the yield of the purification process by more than 10, 9, 8, 7, 6, 5, 4, 3, 2, 1, 0.5 or 0.1% relative to a solution being completely free of particles.

The final transferrin-containing solution obtained from the process of the first, second or third aspect of the invention may or may not be subjected to one or more further steps such as purification steps. Such further steps may be carried out prior to one or more of the steps of the first, second or third aspects of the invention. For example, the first and second aspects of the invention may be separated by one or more of the further steps.

The one or more further purification steps may be selected from: anion exchange chromatography, cation exchange chromatography, buffer exchange, concentration, dilution, removal of metal ions such as iron ions, dialysis, diafiltration, pH-adjustment, (for example with solution comprising tetraborate and a base, such as potassium tetraborate and sodium hydroxide, particularly 250 mM potassium tetraborate and 0.5 M sodium hydroxide), treatment with a reducing agent, discolouration treatment (e.g. with charcoal) for example to remove yeast-derived and/or matrix-derived colourants, heating (including sterilisation), cooling, conditioning, ultrafiltration, cell separation techniques, such as centrifugation, filtration (e.g. cross-flow filtration, expanded bed chromatography and the like) methods of cell breakage, including beadmilling, sonication, enzymatic exposure and the like, ammonium sulphate precipitation, ethanol precipitation, acid extraction, solvent extraction, phosphocellulose chromatography, hydrophobic interaction chromatography, affinity chromatography, hydroxyapatite chromatography, lectin chromatography, metal affinity/chelating chromatography, high performance liquid chromatography (“HPLC”), reverse phase HPLC, conductivity adjustment, alkaline precipitation of the transferrin to a lipophilic phase and the like. Ultrafiltration is particularly preferred. ‘Conditioning’ includes adjusting one or more of pH and conductivity and/or the addition of solid and/or ingredients. Ultrafiltration may use a nominal molecular weight cut off of 10 000. Transferrin recovered from the process of the first, second or third aspect of the invention may be pH adjusted to from pH 6.7 to 7.3, for example by the addition of acetic acid. The pH-adjusted transferrin may then be concentrated and/or diafiltered against a NaCl solution. At the end of diafilitration the retentate may be further concentrated.

Any one or more of the above mentioned techniques may or may not be used to further purify the thus isolated protein to a commercially or industrially acceptable level of purity. By commercially or industrially acceptable level of purity, we include the provision of the transferrin protein in which other material (for example, one or more contaminants) are present at a level of less than 50%, 40%, 30%, 20%, 10%, 5%, 4%, 3%, 2%, 1%, 0.5%, 0.1%, 0.01%, 0.001%, 0.0001%, 0.00001%, or 0.000001% and, most preferably at a level of 0%.

A commercially or industrially acceptable level of purity may be obtained by a relatively crude purification method by which the protein product of choice is put into a form suitable for its intended purpose. A protein preparation that has been purified to a commercially or industrially acceptable level of purity may, in addition to the protein product of choice, also comprise, for example, cell culture components such as host cells or debris derived therefrom. Alternatively, high molecular weight components (such as host cells or debris derived therefrom) may or may not be removed (such as by filtration or centrifugation) to obtain a composition comprising the protein product of choice and, optionally, a functionally acceptable level of low molecular weight contaminants derived from the cell culture process.

The protein may or may not be purified to achieve a pharmaceutically acceptable level of purity. A protein has a pharmaceutically acceptable level of purity if it is essentially pyrogen free and can be used for its intended purpose and hence be administered in a pharmaceutically efficacious amount without causing medical effects not associated with the activity of the protein.

The transferrin product may be provided at a concentration of at least 10⁴ g.L⁻¹, 10⁻³ g.L⁻¹, 0.01 g.L⁻¹, 0.02 g.L⁻¹, 0.03 g.L⁻¹, 0.04 g.L⁻¹, 0.05 g.L⁻¹, 0.06 g.L⁻¹,0.07 g.L⁻¹, 0.08 g.L⁻¹, 0.09 g.L⁻¹, 0.1 g.L⁻¹, 0.2 g.L⁻¹, 0.3 g.L⁻¹, 0.4 g.L⁻¹, 0.5 g.L⁻¹, 0.6 g.L⁻¹, 0.7 g.L⁻¹, 0.8 g.L⁻¹, 0.9 g.L⁻¹, 1 g.L⁻¹, 2 g.L⁻¹, 3 g.L⁻¹, 4 g.L⁻¹, 5 g.L⁻¹, 6 g.L⁻¹, 7 g.L⁻¹, 8 g.L⁻¹, 9 g.L⁻¹, 10 g.L⁻¹, 15 g.L⁻¹, 20 g.L⁻¹, 25 g.L^{-1 1}, 30 g.L⁻¹, 40 g.L⁻¹, 50 g.L⁻¹, 60 g.L⁻¹, 70 g.L⁻¹, 70 g.L⁻¹, 90 g.L⁻¹, 100 g.L⁻¹, 150 g.L⁻¹, 200 g.L⁻¹, 250 g.L⁻¹, 300 g.L⁻¹, 350 g.L⁻¹, 400 g.L⁻¹, 500 g.L⁻¹, 600 g.L⁻¹, 700 g.L⁻¹, 800 g.L⁻¹, 900 g.L⁻¹, 1000 g.L⁻¹.

The transferrin-containing solution obtained by the process of the first, second or third aspect of the invention may be further purified and/or formulated for intravenous administration to a human and/or for provision as a cell culture ingredient and/or sterilised and/or placed into a final container.

It is preferred that the transferrin-containing solution obtained by the first, second or third aspect of the invention is suspended in a liquid, for example a solution of a buffer, suitable for final use of the transferrin. Such solutions include sodium chloride solutions, for example 145 mM NaCl.

The process of the first, second or third aspects of the invention may be preceded by one or more of the following steps: fermentation, primary separation, centrate conditioning, affinity separation, metal chelating separation, hydrophobic separation, liquid/liquid extraction, precipitation, ultrafiltration, size exclusion separation and/or mixed mode separation.

The transferrin may or may not be holoized prior to and/or following the cationic chromatographic step and/or the anionic chromatographic step of the first, second or third aspects of the invention. The transferrin may or may not be apoized prior to and/or following the cationic chromatographic step and/or the anionic chromatographic step of the first, second or third aspects of the invention. That is, the transferrin which is subjected to the purification process may be apo-transferrin, partially iron-saturated transferrin or holotransferrin.

Holoization and apoization techniques are known in the art. Transferrin may be holoized by the addition of sodium bicarbonate and iron (for example in the form of ammonium iron citrate) to about 2 moles of Fe³⁺per mole of transferrin and incubating at ambient temperature (e.g. about 20 to 25° C.) for at least one hour. Transferrin may be apoized at low pH in the presence of a chelating agent. For example transferrin may be apoized by the addition of 1 part transferrin solution to 9 parts of apoization solution and incubating the resultant mixture, with stirring, overnight at about 4° C. Apoization solution may comprise 100 mM sodium acetate, 100 mM sodium citrate, 10 mM EDTA at pH 4.5.

The relatively impure transferrin may be obtained from a blood source or from a recombinant source such as prokaryotic (e.g. bacterial) or eukaryotic (e.g. fungal, animal, insect or plant). A preferred source of the relatively impure solution of transferrin is a microbial cell, such as a fungal cell (preferably a yeast cell such as Saccharomyces (e.g. S. cerevisiae), Pichia (e.g. P. pastoris) or Kluyveromyces (e.g. K. lactis)) or a bacterial cell (e.g. a Bacillus or Escherichia coli), a mammalian cell (e.g. Chinese Hamster Ovary (CHO) or Baby Hamster Kidney (BHK) cell) or from blood or a blood fraction such as serum. The blood or blood fraction may be human or animal derived (such as bovine derived).

The ‘transferrin’ of the invention may be any protein with iron binding capacity which belongs to the transferrin family as described, e.g., by Lambert et al., Comparative Biochemistry and Physiology, Part B, 142 (2005), 129-141, and by Testa, Proteins of iron metabolism, CRC Press, 2002; Harris & Aisen, Iron carriers and iron proteins, Vol. 5, Physical Bioinorganic Chemistry, VCH, 1991.

Examples of transferrin family proteins are serum transferrin, ovotransferrin, melanotransferrin and lactoferrin and their derivatives and variants, such as mutant transferrins (Mason et al., (1993) Biochemistry, 32, 5472; Mason et al., (1998), Biochem. J., 330, 35), truncated transferrins, transferrin lobes (Mason et al., (1996) Protein Expr. Purif., 8, 119; Mason et al., (1991) Protein Expr. Purif., 2, 214), mutant lactoferrins, truncated lactoferrins, lactoferrin lobes, mutant ovotransferrin, truncated ovotransferrin, melanotransferrin lobes, truncated melanotransferrin or fusions of any of the above to other peptides, polypeptides or proteins (Shin et al., (1995) Proc. Natl. Acad. Sci. USA, 92, 2820; Ali et al., (1999) J. Biol. Chem., 274, 24066; Mason et al., (2002) Biochemistry, 41, 9448). Serum transferrins are preferred, particularly a human serum transferrin (HST) having the amino acid sequence of SEQ ID NO: 1 with 679 amino acids, also called the Cl variant (Accession number NP_{—}001054). Lactoferrins are preferred, particularly a human lactoferrin having the amino acid sequence of SEQ ID NO: 2 with 691 amino acids. Melanotransferrins are preferred, partitularly human melanotransferrin having the amino acid sequence residue 20-738 of SEQ ID NO: 3.

The term ‘transferrin’ also includes both natural and engineered variants of transferrins for example fusions and conjugations of transferrins and conjugation-competent transferrins such as ‘thio-transferrins’. Examples are described in WO2008/152140 and PCT/EP2008/060482, both of which are hereby incorporated by reference. Conjugations of transferrin, for example ‘thio-transferrins’ may be prepared according to PCT/EP2008/060482 and “Protein Formulation and Delivery”, E. J. McNally (Ed.), published by Marcel Dekker Inc. New York 2000 and “Rational Design of Stable Protein Formulations—Theory and Practice”; J. F. Carpenter and M. C. Manning (Ed.) Pharmaceutical Biotechnology Vol 13. Kluwer Academic/Plenum Publishers, New York 2002, Yazdi and Murphy, (1994) Cancer Research 54, 6387-6394, Widera et al., (2003) Pharmaceutical Research 20, 1231-1238; Lee et al., (2005) Arch. Pharm. Res. 28, 722-729. The transferrin of or purified by the claimed invention may or may not be a natural or engineered variant of transferrin as described herein. For example, the transferrin may or may not have one or more thiol groups available for conjugation to another molecule.

| [SEQ ID No. 1] | |
|---|---|
| VPDKTVRWCA VSEHEATKCQ SFRDHMKSVI PSDGPSVACV KKASYLDCIR AIAANEADAV | 60 |
| TLDAGLVYDA YLAPNNLKPV VAEFYGSKED PQTFYYAVAV VKKDSGFQMN QLRGKKSCHT | 120 |
| GLGRSAGWNI PIGLLYCDLP EPRKPLEKAV ANFFSGSCAP CADGTDFPQL CQLCPGCGCS | 180 |
| TLNQYFGYSG AFKCLKDGAG DVAFVKHSTI FENLANKADR DQYELLCLDN TRKPVDEYKD | 240 |
| CHLAQVPSHT VVARSMGGKE DLIWELLNQA QEHFGKDKSK EFQLFSSPHG KDLLFKDSAH | 300 |
| GFLKVPPRMD AKMYLGYEYV TAIRNLREGT CPEAPTDECK PVKWCALSHH ERLKCDEWSV | 360 |
| NSVGKIECVS AETTEDCIAK IMNGEADAMS LDGGFVYIAG KCGLVPVLAE NYNKSDNCED | 420 |
| TPEAGYFAVA VVKKSASDLT WDNLKGKKSC HTAVGRTAGW NIPMGLLYNK INHCRFDEFF | 480 |
| SEGCAPGSKK DSSLCKLCMG SGLNLCEPNN KEGYYGYTGA FRCLVEKGDV AFVKHQTVPQ | 540 |
| NTGGKNPDPW AKNLNEKDYE LLCLDGTRKP VEEYANCHLA RAPNHAVVTR KDKEACVHKI | 600 |
| LRQQQHLFGS NVTDCSGNFC LFRSETKDLL FRDDTVCLAK LHDRNTYEKY LGEEYVKAVG | 660 |
| NLRKCSTSSL LEACTFRRP | 679 |
| SEQ ID No. 2] | |
| GRRRSVQWCA VSQPEATKCF QWQRNMRKVR GPPVSCIKRD SPIQCIQAIA ENRADAVTLD | 60 |
| GGFIYEAGLA PYKLRPVAAE VYGTERQPRT HYYAVAVVKK GGSFQLNELQ GLKSCHTGLR | 120 |
| RTAGWNVPIG TLRPFLNWAG PPEPIEAAVA RFFSASCVPG ADKGQFPNLC RLCAGTGENK | 180 |
| CAFSSQEPYF SYSGAFKCLR DGAGDVAFIR ESTVFEDLSD EAERDEYELL CPDNTRKPVD | 240 |
| KFKDCHLARV PSHAVVARSV NGKEDAIWNL LRQAQEKFGK DKSPKFQLFG SPSGQKDLLF | 300 |
| KDSAIGFSRV PPRIDSGLYL GSGYFTAIQN LRKSEEEVAA RRARVVWCAV GEQELRKCNQ | 360 |
| WSGLSEGSVT CSSASTTEDC IALVLKGEAD AMSLDGGYVY TAGKCGLVPV LAENYKSQQS | 420 |
| SDPDPNCVDR PVEGYLAVAV VRRSDTSLTW NSVKGKKSCH TAVDRTAGWN IPMGLLFNQA | 480 |
| GSCKFDEYFS QSCAPGSDPR SNLCALCIGD EQGENKCVPN SNERYYGYTG AFRCLAENAG | 540 |
| DVAFVKDVTV LQNTDGNNNE AWAKDLKLAD FALLCLDGKR KPVTEARSCH LAMAPNHAVV | 600 |
| SRMDKVERLK QVLLHQQAKF GRNGADCPDK FCLFQSETKN LLFNDNTECL ARLHGKTTYE | 660 |
| KYLGPQYVAG ITNLKKCSTS PLLEACEFLR K | 691 |
| [SEQ ID No. 3] | |
| MRGPSGALWL LLALRTVLGG MEVRWCATSD PEQHKCGNMS EAFREAGIQP SLLCVRGTSA | 60 |
| DHCVQLIAAQ EADAITLDGG AIYEAGKEHG LKPVVGEVYD QEVGTSYYAV AVVRRSSHVT | 120 |
| IDTLKGVKSC HTGINRTVGW NVPVGYLVES GRLSVMGCDV LKAVSDYFGG SCVPGAGETS | 180 |
| YSESLCRLCR GDSSGEGVCD KSPLERYYDY SGAFRCLAEG AGDVAFVKHS TVLENTDGKT | 240 |
| LPSWGQALLS QDFELLCRDG SRADVTEWRQ CHLARVPAHA VVVRADTDGG LIFRLLNEGQ | 300 |
| RLFSHEGSSF QMFSSEAYGQ KDLLFKDSTS ELVPIATQTY EAWLGHEYLH AMKGLLCDPN | 360 |
| RLPPYLRWCV LSTPEIQKCG DMAVAFRRQR LKPEIQCVSA KSPQHCMERI QAEQVDAVTL | 420 |
| SGEDIYTAGK TYGLVPAAGE HYAPEDSSNS YYVVAVVRRD SSHAFTLDEL RGKRSCHAGF | 480 |
| GSPAGWDVPV GALIQRGFIR PKDCDVLTAV SEFFNASCVP VNNPKNYPSS LCALCVGDEQ | 540 |
| GRNKCVGNSQ ERYYGYRGAF RCLVENAGDV AFVRHTTVFD NTNGHNSEPW AAELRSEDYE | 600 |
| LLCPNGARAE VSQFAACNLA QIPPHAVMVR PDTNIFTVYG LLDKAQDLFG DDHNKNGFKM | 660 |
| FDSSNYHGQD LLFKDATVRA VPVGEKTTYR GWLGLDYVAA LEGMSSQQCS GAAAPAPGAP | 720 |
| LLPLLLPALA ARLLPPAL | 738 |

A particularly preferred transferrin consists of or comprises the amino acid sequence of SEQ ID No. 4:

| [SEQ ID No. 4] | |
|---|---|
| VPDKTVRWCA VSEHEATKCQ SFRDHMKSVI PSDGPSVACV KKASYLDCIR AIAANEADAV | 60 |
| TLDAGLVYDA YLAPNNLKPV VAEFYGSKED PQTFYYAVAV VKKDSGFQMN QLRGKKSCHT | 120 |
| GLGRSAGWNI PIGLLYCDLP EPRKPLEKAV ANFFSGSCAP CADGTDFPQL CQLCPGCGCS | 180 |
| TLNQYFGYSG AFKCLKDGAG DVAFVKHSTI FENLANKADR DQYELLCLDN TRKPVDEYKD | 240 |
| CHLAQVPSHT VVARSMGGKE DLIWELLNQA QEHFGKDKSK EFQLFSSPHG KDLLFKDSAH | 300 |
| GFLKVPPRMD AKMYLGYEYV TAIRNLREGT CPEAPTDECK PVKWCALSHH ERLKCDEWSV | 360 |
| NSVGKIECVS AETTEDCIAK IMNGEADAMS LDGGFVYIAG KCGLVPVLAE NYNKADNCED | 420 |
| TPEAGYFAVA VVKKSASDLT WDNLKGKKSC HTAVGRTAGW NIPMGLLYNK INHCRFDEFF | 480 |
| SEGCAPGSKK DSSLCKLCMG SGLNLCEPNN KEGYYGYTGA FRCLVEKGDV AFVKHQTVPQ | 540 |
| NTGGKNPDPW AKNLNEKDYE LLCLDGTRKP VEEYANCHLA RAPNHAVVTR KDKEACVHKI | 600 |
| LRQQQHLFGS NVADCSGNFC LFRSETKDLL FRDDTVCLAK LHDRNTYEKY LGEEYVKAVG | 660 |
| NLRKCSTSSL LEACTFRRP | 679 |

Preferably the transferrin which undergoes purification does not contain a leader sequence. For example, a transferrin may be produced with a leader sequence and the leader sequence may or may not be removed prior to the purification. Alternatively, the transferrin may be produced without the leader sequence. Examples of transferrin sequences without a leader sequence are given in SEQ ID No. 1 and SEQ ID No. 4.

The transferrin may be modified relative to a naturally occurring transferrin or to a known engineered variant of transferrin. For example, the transferrin may consist of or comprise an amino acid sequence which has at least 25% identity to SEQ ID NO: 1, SEQ ID No. 2 or SEQ ID No. 3 or SEQ ID No. 4, particularly at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%. More particularly, it may have 100% identity to SEQ ID No. 1, SEQ ID No. 2, SEQ ID No. 3 or most particularly SEQ ID No. 4.

The amino acid sequence of the transferrin family protein may have a length of at least 167 amino acids, particularly at least 300 amino acids, 630 amino acids, 691 amino acids, 694 amino acids or 695 amino acids. The length is typically at most 1274 amino acids, particularly at most 819 amino acids, at most 722 amino acids, at most 717 amino acids or at most 706 amino acids. The length may particularly be 679 amino acids. It may be a vertebrate transferrin with 691-717 amino acids or a mammalian transferrin with a length of 695-706 amino acids.

The transferrin family protein, particularly one having a length of 694-706 amino acids, generally contains two domains (called the N- and C-lobes), each having around 331-341 residues and each binding one atom of Fe (III) and one carbonate anion, connected by an inter-lobe linker of about 7 residues. Typically, each iron is coordinated to four conserved amino acid residues: one Asp, two Tyr and one H is, and the carbonate anion is bound to an Arg and a Thr.

Preferably, the transferrin family protein purified in the invention has at least 40% or at least 60% identity to full-length HST (residues 1-679 of SEQ ID NO: 1) or to the C-lobe of HST (residues 339-679 of SEQ ID NO. 1). The primary receptor-recognition site of HST for the TfR is in the C-lobe, and proteolytically isolated HST C-lobe is able to deliver ferric iron to cells.

The human serum transferrin may be the variant designated TfC₁, TfC₂ or TfC₃.

A number of proteins are known to exist within the transferrin family and a non-exclusive list is shown below. The list indicates the full length of the sequences, including the mature protein and the leader sequence. Each of these transferrins, and variants thereof, may or may not be purified by the process of the invention.

TABLE 1
Examples of transferrins

| | | | | Iden- | |
|---|---|---|---|---|---|
| | | | | tity to | |
| | | Common | Accession | SEQ ID | |
| Protein | Species | Name | Number | NO: 1 | Length |
| Transferrin | Homo sapiens | Human | NP_001054 | 100% | 698 aa |
| Transferrin | Canis lupus familiaris | Dog | XP_864515 | 73.2 | 706 aa |
| Transferrin | Mus musculus | House mouse | NP_598738 | 73.7 | 697 aa |
| Transferrin | Rattus norvegicus | Norway rat | NP_001013128 | 73.6 | 698 aa |
| Transferrin | Sus scrofa | Pig | CAA30943 | 71.6 | 696 aa |
| Transferrin | Oryctolagus cuniculus | Rabbit | AAB94136 | 79.0 | 695 aa |
| Transferrin | Equus caballus | Horse | NP_001075415 | 73.7 | 706 aa |
| Transferrin | Bos taurus | Cattle | NP_803450 | 70.1 | 704 aa |
| Transferrin | Gallus gallus | Chicken | NP_990635 | 53.1 | 705 aa |
| Transferrin | Marmota monax | Woodchuck | AAP37129 | | 694 aa |
| Transferrin | Xenopus laevis | African clawed | NP_001079812 | | 717 aa |
| | | frog | | | |
| Transferrin | Xenopus tropicalis | African clawed | NP_001027487 | 49.6 | 703 aa |
| | | frog | | | |
| Transferrin | Chamaeleo calyptratus | Veiled Chame- | CAK18229 | | 710 aa |
| | | leons | | | |
| Transferrin | Lacerta agilis | Sand Lizard | CAK18228 | | 714 aa |
| Transferrin | Eublepharis | Leopard gecko | CAK18227 | | 703 aa |
| | macularius | | | | |
| Transferrin | Pogona vitticeps | Central | CAK18226 | | 702 aa |
| | | bearded | | | |
| | | dragon | | | |
| Transferrin | Anolis sagrei | Brown anole | CAK18225 | | 710 aa |
| Transferrin | Lamprophis | African House | CAK18223 | | 711 aa |
| | fuliginosus | Snake | | | |
| Transferrin | Natrix natrix | Grass Snake | CAK18221 | 50.0 | 710 aa |
| Transferrin | Oncorhynchus | Rainbow trout | BAA84103 | | 691 aa |
| | mykiss | | | | |
| Transferrin | Oryzias latipes | Japanese | BAA10901 | | 690 aa |
| | | medaka | | | |
| Transferrin | Oreochromis | Nile tilapia | ABB70391 | | 694 aa |
| | niloticus | | | | |
| Transferrin | Oncorhynchus | Chinook | AAF03084 | | 672 aa |
| | tshawytscha | salmon | | | |
| Transferrin | Gadus morhua | Atlantic cod | AAB08440 | | 642 aa |
| Transferrin | Salmo trutta | Brown trout | BAA84102 | | 691 aa |
| Transferrin | Salvelinus | Lake trout | BAA84101 | | 691 aa |
| | namaycush | | | | |
| Transferrin | Salvelinus fontinalis | Brook trout | BAA84100 | | 691 aa |
| Transferrin | Salvelinus pluvius | Japanese fish | BAA84099 | | 691 aa |
| Transferrin | Oncorhynchus | Cherry salmon | BAA84098 | | 691 aa |
| | masou | | | | |
| Transferrin | Oncorhynchus | Amago | BAA84097 | | 691 aa |
| | rhodurus | | | | |
| Transferrin | Oncorhynchus nerka | Sockeye | BAA84096 | 50.2 | 691 aa |
| | | salmon | | | |
| Transferrin | Paralichthys | Bastard halibut | BAA28944 | | 685 aa |
| | olivaceus | | | | |
| Transferrin | Oncorhynchus | Coho salmon | BAA13759 | | 687 aa |
| | kisutch | | | | |
| Transferrin | Oreochromis aureus | Tilapia (cichlid) | CAC59954 | | 167 aa |
| Transferrin | Danio rerio | Zebrafish | DAA01798 | | 675 aa |
| Transferrin | Pagrus major | Red seabream | AAP94279 | | 691 aa |
| Melanotransferrin | Homo sapiens | Human | NP_005920 | 45.7 | 738 aa |
| Meanotransferrin | Canis lupus familiaris | Dog | XP_545158 | 42.3 | 1193 aa |
| Melanotransferrin | Mus musculus | House mouse | NP_038928 | 44.4 | 738 aa |
| Melanotransferrin | Rattus norvegicus | Norway rat | XP_237839 | 44.7 | 738 aa |
| Melanotransferrin | Gallus gallus | Chicken | NP_990538 | 43.6 | 738 aa |
| Lactotransferrin | H. sapiens | Human | NP_002334 | 61.8 | 710 aa |
| Lactotransferrin | Pan troglodytes | Chimpanzee | XP_516417 | 62.0 | 711 aa |
| Lactotransferrin | Canis lupus familiaris | Dog | XP_864480 | 61.9 | 724 aa |
| Lactotransferrin | Mus musculus | House mouse | NP_032548 | 57.6 | 707 aa |
| Lactotransferrin | Rattus norvegicus | Norway rat | XP_236657 | 53.8 | 729 aa |
| Lactoferrin | Sus scrofa | Pig | AAA31059 | 61.1 | 703 aa |
| Lactoferrin | Camelus dromedarius | Arabian camel | CAB53387 | 62.0 | 708 aa |
| Lactoferrin | Equus caballus | Horse | CAA09407 | 63.7 | 695 aa |
| Lactoferrin | Bos taurus | Cattle | AAA30610 | 62.0 | 708 aa |
| Lactoferrin | Bubalus bubalis | Water buffalo | CAA06441 | 62.1 | 708 aa |
| Lactoferrin | Capra hircus | Goat | ABD49106 | 62.2 | 708 aa |
| Lactoferrin | Bos grunniens | Domestic yak | ABD49105 | 62.0 | 708 aa |
| Lactoferrin | Ovis aries | Sheep | AAV92908 | 62.4 | 708 aa |
| Ovotransferrin | Anas platyrhynchos | Mallard duck | P56410 | 54.4 | 686 aa |
| Ovotransferrin | Gallus gallus | Chicken | CAA26040 | 53.1 | 705 aa |

The transferrin may or may not be fused and/or conjugated to a protein or other molecule such as a bioactive compound. The protein or other molecule, such as a bioactive compound, may or may not have a therapeutic and/or diagnostic use. In this context, ‘fusion’ includes a protein in which the transferrin and the other protein are encoded by the same DNA sequence whereas ‘conjugation’ includes a protein in which the transferrin has a protein or other molecule bound to it through any other means, for example through chemical conjugation. US 20030221201 and 20040023334 describe fusion proteins comprising a transferrin protein fused to a therapeutic protein. The transferrin may or may not be both a fused and conjugated transferrin.

The transferrin may or may not be a therapeutic or diagnostic transferrin. A ‘therapeutic transferrin’ is a transferrin which is suitable for use in preventative therapy or for curative therapy, for example treatment of anaemia. A ‘diagnostic transferrin’ is a transferrin which is suitable for use in diagnosis, for example the transferrin may be fused or conjugated to an antibody or to a label such as label or contrast agent suitable for use in imaging. The diagnostic transferrin may be used in vivo or in vitro.

The therapeutic transferrin may comprise a chemotherapy drug for use in cancer chemotherapy. It may be cytostatic or cytotoxic; it may be a tumor-inhibiting agent.

The bioactive compound may be a polypeptide (protein), particularly a recombinant protein pharmaceutical. It may be a chemotherapy or radiotherapy drug used to treat cancers and/or other related diseases.

The bioactive compound may be conjugated to a free thiol of a transferrin. The bioactive compound includes but is not limited to, peptides, polypeptides or proteins (either natural, recombinant, or synthetic). The free thiol may be on a cysteine residue.

A fourth aspect of the invention comprises a process for producing a transferrin comprising expressing a transferrin, for example in cells, particularly in fungal cells such as yeast cells, and purifying the resultant transferrin in accordance with the first, second, or third aspect of the invention.

A fifth aspect of the invention comprises the step of lyophilising the transferrin purifled by the first, second, third or fourth aspect of the invention.

A sixth aspect of the invention provides a purified transferrin obtained or obtainable by a process according to the first, second, third, fourth or fifth aspect of the invention.

A seventh aspect of the invention relates to the use of a transferrin according to the first, second, third, fourth, fifth or sixth aspect of the invention in therapy (preventative and/or curative) or diagnosis. For example, the transferrin may be used in leukaemia treatment to prevent the harmful effects of non-transferrin-bound iron in a patient. The transferrin, for example a fused or conjugated transferrin, may be used in targeting, for example in diagnosis, imaging, localization and/or treatment of tumours.

An eighth aspect of the invention relates to the use of a transferrin according to the first, second, third, fourth, fifth, or sixth aspect of the invention in cell culture such as mammalian cell culture, particularly human cell culture. Cell culture may be at laboratory or industrial scale. The invention also relates to a cell culture medium comprising the transferrin according to the first, second, third, fourth, fifth or sixth aspect of the invention. The invention also includes manufacture of such a cell culture medium.

A ninth aspect of the invention provides a pharmaceutical composition comprising a transferrin purified according to the first, second, third, fourth or fifth aspect of the invention and a pharmaceutically acceptable carrier and/or diluent. Therefore, the purified transferrin may be formulated with a carrier or diluent. The thus formulated transferrin may optionally be presented in a unit dosage form. The carrier(s) or diluent(s) must be “acceptable” in the sense of being compatible with the desired protein. Typically, the carriers or diluents will be water or saline which will be sterile and pyrogen free. The invention also includes provision of a transferrin according to the invention for use as a pharmaceutical ingredient. The transferrin may or may not be fused or conjugated to another molecule which may or may not be a bioactive molecule.

With regards the ‘starting material’ (i.e. the relatively impure transferrin solution from which the transferrin is purified by the process of the invention), the transferrin or fusions of transferrin and another protein or proteins can be prepared by methods know in the art (Sanker, (2004), Genetic Eng. News, 24, 22-28, Schmidt, (2004), Appl. Microbiol. Biotechnol., 65, 363-372) including but not limited to expression in mammalian cell culture (Mason et al., (2004), Protein Expr. Purif., 36, 318-326; Mason et al., (2002), Biochemistry, 41, 9448-9454) from cells lines such as CHO (and its variants), NS0, BHK, HEK293, Vero or PERC6 cells by transformation or transient expression; insect cell culture (Lim et al., (2004) Biotechnol. Prog., 20, 1192-1197); plant cell culture from such plants as Lemna; transgenic animals (Dyck et al., (2003) Trends in Biotechnology, 21, 394-399); transgenic plants (Ma et al., (2003) Nature Reviews Genetics, 4, 794-805); Gram positive and Gram negative bacteria such as Bacillus and Escherichia coli (Steinlein, and Ikeda, (1993), Enzyme Microb. Technol., 15, 193-199); filamentous fungi including but not restricted to Aspergillus spp (EP 238023, U.S. Pat. No. 5,364,770, U.S. Pat. No. 5,578,463, EP184438, EP284603, WO 2000/056900, WO9614413), Trichoderma spp and Fusarium spp (Navalainen et al., (2005), Trends in Biotechnology, 23, 468-473).

Polypeptides which are variants of full-length HST may be expressed recombinantly from baby hamster kidney (BHK) cells (Mason et al., (2004), Protein Expr. Purif., 36, 318-326, Mason et al., (2002), Biochemistry, 41, 9448-9454), D. melanogaster S2 cells (Lim et al., (2004), Biotechnol Prog., 20, 1192-1197) and as a non-N-linked glycosylated mutant from BHK cells (Mason et al., (2001), Protein Expr. Purif., 23, 142-150, Mason et al., (1993), Biochemistry, 32, 5472-5479) and S. cerevisiae (Sargent et al., (2006), Biometals 19, 513-519). The polypeptides which are variants of C-lobe of HST (NTf/2C) may be expressed from BHK cells (Mason et al., (1997), Biochem. J., 326 (Pt 1), 77-85). In one embodiment the host cell is a yeast cell, such as a member of the Saccharomyces, Kluyveromyces, or Pichia genus, such as Sacharomyces cerevisiae, Kluyveromyces lactis, Pichia pastoris (Mason et al., (1996), Protein Expr. Purif., 8, 119-125, Steinlein et al., 1995 Protein Expr. Purif., 6, 619-624), Pichia methanolica (Mayson et al., (2003) Biotechnol. Bioeng., 81, 291-298) and Pichia membranaefaciens, or Zygosaccharomyces rouxii (formerly classified as Zygosaccharomyces bisporus), Zygosaccharomyces bailii, Zygosaccharomyces fermentati, Hansenula polymorpha (also known as Pichia angusta) or Kluyveromyces drosophilarum are preferred.

The host cell may be any type of cell. The host cell may or may not be an animal (such as mammalian, avian, insect, etc.), plant, fungal or bacterial cell. Bacterial and fungal, such as yeast, host cells may or may not be preferred.

Typical prokaryotic vector plasmids are: pUC18, pUC19, pBR322 and pBR329 available from BioRad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

A typical mammalian cell vector plasmid is pSVL available from Pharmacia (Piscataway, NJ, USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Methods well known to those skilled in the art can be used to construct expression vectors containing the coding sequence of a desired protein and, for example appropriate transcriptional or translational controls. One such method involves ligation via cohesive ends. Compatible cohesive ends can be generated on the DNA fragment and vector by the action of suitable restriction enzymes. These ends will rapidly anneal through complementary base pairing and remaining nicks can be closed by the action of DNA ligase.

A further method uses synthetic double stranded oligonucleotide linkers and adaptors. DNA fragments with blunt ends are generated by bacteriophage T4 DNA polymerase or E. coli DNA polymerase I which remove protruding 3′ termini and fill in recessed 3′ ends. Synthetic linkers and pieces of blunt-ended double-stranded DNA which contain recognition sequences for defined restriction enzymes, can be ligated to blunt-ended DNA fragments by T4 DNA ligase. They are subsequently digested with appropriate restriction enzymes to create cohesive ends and ligated to an expression vector with compatible termini. Adaptors are also chemically synthesised DNA fragments which contain one blunt end used for ligation but which also possess one preformed cohesive end. Alternatively a DNA fragment or DNA fragments can be ligated together by the action of DNA ligase in the presence or absence of one or more synthetic double stranded oligonucleotides optionally containing cohesive ends.

Synthetic linkers containing a variety of restriction endonuclease sites are commercially available from a number of sources including Sigma-Genosys Ltd, London Road, Pampisford, Cambridge, United Kingdom.

The transferrin or fusions of transferrin and another protein or proteins will be expressed from a nucleotide sequence, which may or may not contain one or more introns. Additionally the nucleotide sequence may or may not be codon optimised for the host by methods known to the art.

The transferrin or fusions of transferrin and another protein or proteins can be expressed as variants with reduced N-linked glycosylation. Accordingly, in case of human serum transferrin (HST), N413 can be changed to any amino acid, preferably, Q, D, E or A; S415 can be changed to any amino acid except S or T, preferably, A; T613 can be changed to any amino acid except S or T, preferably, A; N611 can be changed to any amino acid; or combinations of the above. Where the transferrin is not HST, reduction in N-glycosylation can be achieved by similar modification to the protein primary sequence. For clarity, the transferrin or fusions of transferrin and another protein or proteins can be both a transferrin variant of the invention and have reduced N-linked glycosylation.

It may be particularly advantageous to use a host, for example a yeast, deficient in one or more protein mannosyl transferases involved in O-glycosylation of proteins, for instance by disruption of the gene coding sequence. Recombinantly expressed proteins can be subject to undesirable post-translational modifications by the producing host cell. The mannosylated transferrin would be able to bind to the lectin Concanavalin A. The amount of mannosylated transferrin produced by the yeast can be reduced by using a yeast strain deficient in one or more of the PMT genes (WO 94/04687). The most convenient way of achieving this is to create a yeast which has a defect in its genome such that a reduced level of one of the Pmt proteins is produced. For example, there may or may not be a deletion, insertion or transposition in the coding sequence or the regulatory regions (or in another gene regulating the expression of one of the PMT genes) such that little or no Pmt protein is produced. Alternatively, the yeast could be transformed to produce an anti-Pmt agent, such as an anti-Pmt antibody. Alternatively, the yeast could be cultured in the presence of a compound that inhibits the activity of one of the PMT genes (Duffy et al, “Inhibition of protein mannosyltrans-ferase 1 (PMT1) activity in the pathogenic yeast Candida albicans”, International Conference on Molecular Mechanisms of Fungal Cell Wall Biogenesis, 26-31 Aug. 2001, Monte Verita, Switzerland, Poster Abstract P38; the poster abstract may be viewed at http://www.micro.biol.ethz.ch/cellwall/). If a yeast other than S. cerevisiae is used, disruption of one or more of the genes equivalent to the PMT genes of S. cerevisiae is also beneficial, e.g. in Pichia pastoris or Kluyveromyces lactis. The sequence of PMT1 (or any other PMT gene) isolated from S. cerevisiae may be used for the identification or disruption of genes encoding similar enzymatic activities in other fungal species. The cloning of the PMT1 homologue of Kluyveromyces lactis is described in WO 94/04687.

The yeast may or may not also have a deletion of the HSP150 and/or YAP3 genes as taught respectively in WO 95/33833 and WO 95/23857.

The transferrin may be produced by recombinant expression and secretion. Where the expression system (i.e. the host cell) is yeast, such as Saccharomyces cerevisiae, suitable promoters for S. cerevisiae include those associated with the PGK1 gene, GAL1 or GAL10 genes, TEF1, TEF2, PYK1, PMA1, CYC1, PHO5, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, α-mating factor pheromone, a-mating factor pheromone, the PRB1 promoter, the PRA1 promoter, the GPD1 promoter, and hybrid promoters involving hybrids of parts of 5′ regulatory regions with parts of 5′ regulatory regions of other promoters or with upstream activation sites (e.g. the promoter of EP-A-258 067). Suitable transcription termination signals are well known in the art. Where the host cell is eukaryotic, the transcription termination signal is preferably derived from the 3′ flanking sequence of a eukaryotic gene, which contains proper signals for transcription termination and polyadenylation. Suitable 3′ flanking sequences may, for example, be those of the gene naturally linked to the expression control sequence used, i.e. may correspond to the promoter. Alternatively, they may be different. In that case, and where the host is a yeast, preferably S. cerevisiae, then the termination signal of the S. cerevisiae ADH1, ADH2, CYC1, or PGK1 genes are preferred.

It may be beneficial for the promoter and open reading frame of the gene encoding the recombinant protein comprising the sequence of a transferrin mutant to be flanked by transcription termination sequences so that the transcription termination sequences are located both upstream and downstream of the promoter and open reading frame, in order to prevent transcriptional read-through into any neighbouring genes, such as 2 μm genes, and vice versa.

In one embodiment, the favoured regulatory sequences in yeast, such as Saccharomyces cerevisiae, include: a yeast promoter (e.g. the Saccharomyces cerevisiae PRB1 promoter), as taught in EP 431 880; and a transcription terminator, preferably the terminator from Saccharomyces ADH1, as taught in EP 60 057.

It may be beneficial for the non-coding region to incorporate more than one DNA sequence encoding a translational stop codon, such as UAA, UAG or UGA, in order to minimise translational read-through and thus avoid the production of elongated, non-natural fusion proteins. The translation stop codon UAA is preferred.

It is preferred that the recombinant protein comprising the sequence of a transferrin mutant is secreted. In that case, a sequence encoding a secretion leader sequence may be included in the open reading frame. Thus, a polynucleotide according to the present invention may comprise a sequence that encodes a recombinant protein comprising the sequence of a transferrin mutant operably linked to a polynucleotide sequence that encodes a secretion leader sequence. Leader sequences are usually, although not necessarily, located at the N-terminus of the primary translation product of an ORF and are generally, although not necessarily, cleaved off the protein during the secretion process, to yield the “mature” protein. Thus, in one embodiment, the term “operably linked” in the context of leader sequences includes the meaning that the sequence that encodes a recombinant protein comprising the sequence of a transferrin mutant is linked, at its 5′ end, and in-frame, to the 3′ end of a polynucleotide sequence that encodes a secretion leader sequence. Alternatively, the polynucleotide sequence that encodes a secretion leader sequence may be located, in-frame, within the coding sequence of the recombinant protein comprising the sequence of a transferrin mutant, or at the 3′ end of the coding sequence of the recombinant protein comprising the sequence of a transferrin mutant.

Numerous natural or artificial polypeptide leader sequences (also called secretion pre regions and pre/pro regions) have been used or developed for secreting proteins from host cells. Leader sequences direct a nascent protein towards the machinery of the cell that exports proteins from the cell into the surrounding medium or, in some cases, into the periplasmic space.

For production of proteins in eukaryotic species such as the yeasts Saccharomyces cerevisiae, Zygosaccharomyces species, Kluyveromyces lactis and Pichia pastoris, a secretion leader sequence may be used. This may comprise a signal (pre) sequence or a prepro leader sequence. Signal sequences are known to be heterogeneous in their amino acid sequence (Nothwehr and Gordon 1990, Bioessays 12, 479-484, or Gierasch 1989, Biochemistry 28, p923-930). In essence, signal sequences are generally N-terminally located, have a basic n-region, a hydrophobic h-region and a polar c-region. As long as this structure is retained the signal sequence will work, irrespective of the amino acid composition. How well they work, i.e. how much mature protein is secreted, depends upon the amino acid sequence. Accordingly, the term “signal peptide” is understood to mean a presequence which is predominantly hydrophobic in nature and present as an N-terminal sequence of the precursor form of an extracellular protein expressed in yeast. The function of the signal peptide is to allow the expressed protein to be secreted to enter the endoplasmic reticulum. The signal peptide is normally cleaved off in the course of this process. The signal peptide may be heterologous or homologous to the yeast organism producing the protein. Known leader sequences include those from the S. cerevisiae acid phosphatase protein (Pho5p) (see EP 366 400), the invertase protein (Suc2p) (see Smith et al. (1985) Science, 229, 1219-1224) and heat-shock protein-150 (Hsp150p) (see WO 95/33833). Additionally, leader sequences from the S. cerevisiae mating factor alpha-1 protein (MFα-1) and from the human lysozyme and human serum albumin (HSA) protein have been used, the latter having been used especially, although not exclusively, for secreting human albumin. WO 90/01063 discloses a fusion of the MFα-1 and HSA leader sequences. In addition, the natural transferrin leader sequence may or may not be used to direct secretion of the recombinant protein comprising the sequence of a transferrin mutant.

The skilled person will appreciate that any suitable plasmid may be used, such as a centromeric plasmid. The examples provide suitable plasmids (centromeric YCplac33-based vectors) for use to transform yeast host cells of the present invention. Alternatively, any other suitable plasmid may be used, such as a yeast-compatible 2 μm-based plasmid.

Plasmids obtained from one yeast type can be maintained in other yeast types (Irie et al, 1991, Gene, 108(1), 139-144; Irie et al, 1991, Mol. Gen. Genet., 225(2), 257-265). For example, pSR1 from Zygosaccharomyces rouxii can be maintained in Saccharomyces cerevisiae. In one embodiment the plasmid may or may not be a 2 μm-family plasmid and the host cell will be compatible with the 2 μm-family plasmid used (see below for a full description of the following plasmids). For example, where the plasmid is based on pSR1, pSB3 or pSB4 then a suitable yeast cell is Zygosaccharomyces rouxii; where the plasmid is based on pSB1 or pSB2 then a suitable yeast cell is Zygosaccharomyces baillis; where the plasmid is based on pSM1 then a suitable yeast cell is Zygosaccharomyces fermentati; where the plasmid is based on pKD1 then a suitable yeast cell is Kluyveromyces drosophilarum; where the plasmid is based on pPM1 then a suitable yeast cell is Pichia membranaefaciens; where the plasmid is based on the 2 μm plasmid then a suitable yeast cell is Saccharomyces cerevisiae or Saccharomyces carlsbergensis. Thus, the plasmid may be based on the 2 μm plasmid and the yeast cell may be Saccharomyces cerevisiae. A 2 μm-family plasmid can be said to be “based on” a naturally occurring plasmid if it comprises one, two or preferably three of the genes FLP, REP1 and REP2 having sequences derived from that naturally occurring plasmid.

Useful yeast episomal plasmid vectors are pRS403-406 and pRS413-416 and are generally available from Stratagene Cloning Systems (La Jolla, Calif. 92037, USA), YEp24 (Botstein, D., et al. (1979) Gene 8, 17-24), and YEplac122, YEplac195 and YEplac181 (Gietz, R. D. and Sugino. A. (1988) Gene 74, 527-534). Other yeast plasmids are described in WO 90/01063 and EP 424 117, as well as the “disintegration vectors of EP-A-286 424 and WO2005061719. Plasmids pRS403, pRS404, pRS405 and pRS406 are Yeast Integrating plasmids (Ylps) and incorporate the yeast selectable markers HIS3, TRP1, LEU2 and URA3, as are Ylplac204, Ylplac211 and Ylplac128 (Gietz, R. D. and Sugino. A. (1988) Gene 74, 527-534). Plasmids pRS413-416 are Yeast Centromere plasmids (YCps) as are YCplac22, YCplac33 and YCplac111 (Gietz, R. D. and Sugino. A. (1988) Gene 74, 527-534).

The processes of the present invention can be used to obtain highly purified trans-ferrin from a relatively impure transferrin solution from a number of sources such as blood products and eukaryotic or prokaryotic cell culture. However, the processes are particularly applicable to purifying recombinant transferrin (rTf), particularly from a yeast such as S. cerevisiae. The transferrin produced in accordance with the invention may be any mammalian transferrin such as bovine transferrin, but it is preferably human transferrin or a derivative thereof.

Various references are cited herein, the disclosures of which are incorporated by reference in their entireties.

With reference to the claims of this specification, the invention also provides:

i. A process according to any of claims 1 to 27 wherein the final transferrin-containing solution obtained thereby is or is not subsequently subjected to one or more further purification steps.

ii. A process according to any of claims 1 to 27 and/or (i), wherein the one or more further purification steps is/are selected from: ammonium sulphate precipitation, ethanol precipitation, acid extraction, solvent extraction, anion exchange chromatography, cation exchange chromatography, phosphocellulose chromatography, hydrophobic interaction chromatography, affinity chromatography, hydroxyapatite chromatography, lectin chromatography, metal affinity/chelating chromatography, concentration, dilution, pH adjustment, diafiltration, ultrafiltration, high performance liquid chromatography (“HPLC”), reverse phase HPLC, conductivity adjustment, removal of matrix-derived dye, removal of yeast-derived colourants and alkaline precipitation of the transferrin to a lipophilic phase.

iii. A process according to any of claims 1 to 27 or any of (i) to (ii) wherein the transferrin-containing solution obtained thereby is or is not subsequently further purified by ultrafiltration.

iv. A process according to any of claims 1 to 27 or any of (i) to (iii) wherein the transferrin-containing solution obtained thereby is or is not further purified and/or formulated for intravenous administration to a human and/or for use as a cell culture ingredient and/or sterilised and/or placed into a final container.

v. A process according to any of claims 1 to 27 or any of (i) to (iv) wherein the transferrin-containing solution obtained thereby is or is not suspended in a liquid suitable for final use of the transferrin.

vi. A process according to any of claims 1 to 27 or any of (i) to (v) in which the transferrin is or is not holoized prior to and/or following the anionic chromatographic step and/or the cationic chromatographic step.

vii. A process according to any of claims 1 to 27 or any of (i) to (vi) in which the transferrin is or is not apoized prior to and/or following the anionic chromatographic step and/or the cationic chromatographic step.

viii. A process according to any of claims 1 to 27 or any of (i) to (vii) in which the relatively impure solution of transferrin is or is not obtained from a microbial cell, such as a fungal cell (preferably a yeast cell such as Saccharomyces, Pichia or Kluyveromyces) or a bacterial cell, a mammalian cell or from blood or a blood fraction such as serum.

ix. A process according to any of claims 1 to 27 or any of (i) to (viii) in which the transferrin is or is not a naturally occurring transferrin.

x. A process according to any of claims 1 to 27 or any of (i) to (ix) in which the transferrin is or is not modified relative to a naturally occurring transferrin.

xi. A process according to (x) which the transferrin comprises or does not comprise SEQ ID No. 4.

xii. A process according to any of claims 1 to 27 or any of (i) to (xi) in which the transferrin is or is not a therapeutic transferrin.

xiii. A purified transferrin obtained or obtainable by a process according to any of claims 1 to 27 or any of (i) to (xii).

xiv. A cell culture medium comprising a transferrin according to (xiii).

xv. Use of a transferrin according to (xiv) in the manufacture a cell culture medium.

The invention is described, by way of example only, with reference to the drawings and the following, non-limiting, examples:

### EXAMPLES

### Example 1

### Cation Exchange Low pH/High Salt Wash

Recovery data obtained for washing Sepharose Fast Flow (SP-FF) with nine different wash buffers (pH/sodium chloride concentration combinations)

(a) Low pH/High Salt Wash

Initial Optimization: Twelve runs were performed on an AKTA™ Explorer 100 Air chromatography system (GE Healthcare) on an 8.6 mL column. Washing was carried out using different combinations of pH and salt concentration. Specifically, pH 4.0, 4.5 and 5.1 and sodium chloride concentrations of 0.25, 1.0 and 2.0 M were used. Start material (i.e. solution of about 3.7 mg/ml transferrin) was thawed and conditioned to pH 5.1; 3.5 mS.cm⁻¹ by adding acetic acid. The column was equilibrated at pH 5.0-5.2 and 2.5-3.1 mS.cm⁻¹, conditioned material was loaded at 25 mg transferrin.mL matrix⁻¹ and washed first with buffer of the appropriate pH (i.e. the same pH the wash to be used in wash 2) and no sodium chloride (wash 1). The column was then washed with the low pH/high salt buffer (wash 2) and bound transferrin was eluted with elution buffer (50 mM sodium phosphate, 25 mM sodium chloride, pH 7.0). Load, flow through, wash and eluate fractions were collected, and their transferrin content assayed by GP.HPLC using an area calibration method. This data was used to calculate mass balance and recovery values. Load, wash 2 and eluate fractions were also subjected to yeast antigen (HCP) ELISA analysis, and yeast antigen (HCP) clearance values were calculated.

TABLE 2
Affect of wash conditions on recovery of transferrin in eluate

Wash conditions

| Run number | NaCl (M) | pH | Eluate recovery (%) |
|---|---|---|---|
| 1 | 0.25 | 4.0 | 53.9 |
| 2 | 1.0 | 4.0 | 38.7 |
| 3 | 2.0 | 4.0 | 48.4 |
| 4 | 0.25 | 4.5 | 87.2 |
| 5 | 1.0 | 4.5 | 76.3 |
| 6 | 2.0 | 4.5 | 94.0 |
| 7 | 0.25 | 4.5 | 82.1 |
| 8 | 1.0 | 4.5 | 74.1 |
| 9 | 2.0 | 4.5 | 79.7 |
| 10 | 0.25 | 5.1 | 15.2 |
| 11 | 1.0 | 5.1 | 15.9 |
| 12 | 2.0 | 5.1 | 31.7 |

These data (Table 2) show that, of the conditions tested, pH 4.5 at any sodium chloride concentration gives the best recoveries and that at pH 4.5, 2.0 M sodium chloride is the optimum.

TABLE 3
Mean recovery and yeast antigen (HCP) clearance
data for washing SP-FF with three different pH/sodium
chloride concentration combinations

| | | |
|---|---|---|
| Wash buffer | Mean eluate | Mean yeast antigen |

| NaCl (M) | pH | recovery (%) | (HCP) clearance (fold) |
|---|---|---|---|
| 0.25 | 4.5 | 84.7 | 24.6 |
| 1 | 4.5 | 75.2 | 27.8 |
| 2 | 4.5 | 86.9 | 25.5 |

Data is a mean of two runs for each sodium chloride concentration. Eluate recovery data is from Table 2. This shows that the wash buffer having 2 M sodium chloride at pH 4.5 gives the best eluate recovery/yeast antigen (HCP) clearance combination.

### (b) Further Optimization

This example shows the affect of low pH/high salt wash on yeast antigen (HCP) clearance and transferrin concentration of the eluate.

An SP-FF run was performed on the AKTA Explorer 100 Air on an 8.6 mL column using the pH 4.5, 2 M sodium chloride wash 2 conditions detailed above but with the addition of a third wash step using the equilibration buffer. A further three runs were performed to compare the following three wash regimes:
A: Wash 1 (50 mM sodium acetate pH 5.1)
B: Wash 1 (50 mM sodium acetate pH 4.5); Wash 2 (27 mM sodium acetate, 2 M sodium chloride pH 4.5)
C: Wash 1 (50 mM sodium acetate pH 4.5); Wash 2 (27 mM sodium acetate, 2 M sodium chloride pH 4.5); Wash 3 (50 mM sodium acetate pH 5.1)

Samples from throughout the purifications were subjected to GP.HPLC and ELISA analysis as described above and the data used to calculate transferrin recovery and yeast antigen (HCP) clearance values.

TABLE 4
Recovery and yeast antigen (HCP) clearance data
comparing the three different SP-FF wash regimes

| | | Eluate | Yeast antigen |
|---|---|---|---|
| | | recovery | (HCP) clear- |
| | SP-FF wash regime | (%) | ance (fold) |
| A | Wash 1: 50 mM sodium acetate pH 5.1 | 72.1 | 16.8 |
| B | Wash 1: 27 mM sodium acetate pH 4.5 | 70.9 | 27.6 |
| | Wash 2: 50 mM sodium acetate, 2M | | |
| | sodium chloride pH 4.5 | | |
| C | Wash 1: 50 mM sodium acetate pH 4.5 | 67.5 | 30.1 |
| | Wash 2: 27 mM sodium acetate, 2M | | |
| | sodium chloride pH 4.5 | | |
| | Wash 3: 50 mM sodium acetate pH 5.1 | | |

These data (Table 4) show that the two-wash regime (B) gives a significantly higher yeast antigen (HCP) clearance than the single-wash regime (A) and that the three-wash regime (C) improves on this further with only a relatively small reduction in eluate recovery.

### Example 2

### Affect of Presence of Borate in Anion Exchange Chromatography Load Buffer on the Recovery of Transferrin from the Q-FF Anion Exchange Chromatography

Q-FF matrix was obtained from GE Healthcare. Tetraborate was added to SP-FF eluate, from Example 1 (Table 4, wash regime C), at a concentration equivalent to 100 moles borate per mole rTf (equivalent to 0.7 mM borate in the load) and as a control no borate was added. Chromatography was performed as described above using a 0.66 cm×15 cm bed height (5.13 mL) column at a matrix capacity of 30 mg.mL⁻¹ matrix using Q-FF. Wash was carried out with 15.7 mM potassium tetraborate. Elution was carried out with 150 mM potassium tetraborate. Fractions were collected during load, wash, elution and NaCl clean and the rTf concentration estimated by RP-HPLC and the recoveries tabulated (Table 5). The flow-through (FT) was also analysed and tabulated (Table 5).

TABLE 5
Effect of the absence (A) and presence (B) of borate in
anion exchange chromatography load buffer on the recovery
of transferrin from Q-FF anion exchange chromatography

| | | |
|---|---|---|
| Fraction | Total (mg) | Recovery (%) |

A: 0 mol · mol borate in load

| | | |
|---|---|---|
| Load | 151.7 | |
| FT | 43.1 | 28.4 |
| Wash | 16.4 | 10.8 |
| Elution | 75.6 | 49.8 |
| NaCl | 12.8 | 29.7 |
| Mass Balance | | 119 |

B: 100 mol · mol borate in load

| | | |
|---|---|---|
| Load | 150.9 | |
| FT | 1.7 | 1.1 |
| Wash | 19.4 | 12.8 |
| Elution | 113.6 | 75.3 |
| NaCl | 13.3 | 8.8 |
| Mass Balance | | 98 |

Use of borate in the load gave a higher overall recovery of transferrin in the eluate (75.3% recovery in the presence of borate, 49.8% recovery in the absence of borate). Also, less transferrin was present in the flow through (FT) when the load included borate (1.1% in the presence of borate, 28.4% in the absence of borate).

### Example 3

### Affect of Presence of Borate in Anion Exchange Chromatography Load Buffer on the Recovery of Transferrin from the Capto™ Q Anion Exchange Chromatography

Capto™ Q Matrix was obtained from GE Healthcare. Potassium tetraborate was added to Capto™ Q load as follows, SP-FF eluate from Example 1 (Table 4, wash regime C) was diluted 5.7 fold with a 1:1 dilution of 15.7 mM potassium tetraborate in water. Finally, sufficient water and 27% (w/v) NaOH was added to achieve a conductivity of 3.5 mS.cm⁻¹ and pH 8.8 (equivalent to 824 mol.mol⁻¹, 5.4 mM borate in the load). As a control (no borate) SP-FF eluate was conditioned with water, 27% (w/v) NaOH and 5 M NaCl to achieve pH 8.8 and a conductivity of 3.5 mS.cm⁻¹. Sufficient load was applied at flow rate of 0.7 mL.min⁻¹ onto a 0.66 cm×2 cm bed height (0.68 mL) column to achieve 40 mg.mL⁻¹ matrix. Wash was carried out with 15.7 mM potassium tetraborate. Elution was carried out with 150 mM potassium tetraborate. Fractions were collected during load, wash, elution and NaCl clean and the rTf concentration estimated by RP-HPLC and the recoveries tabulated (Table 6). The flow-through (FT) was also analysed and tabulated (Table 6).

TABLE 6
Effect of the absence (A) and presence (B) of borate in anion
exchange chromatography load buffer on the recovery of transferrin
from Capto ™Q anion exchange chromatography

| | | |
|---|---|---|
| Fraction | Total (mg) | Recovery (%) |

A: 0 mol · mol borate in load

| | | |
|---|---|---|
| Load | 28.0 | |
| FT | 8.8 | 31.3 |
| Wash | 2.5 | 9.0 |
| Elution | 15.0 | 53.6 |
| Mass Balance | | 94 |

B: 825 mol · mol borate in load

| | | |
|---|---|---|
| Load | 27.0 | |
| FT | 3.1 | 11.4 |
| Wash | 1.2 | 4.3 |
| Elution | 21.7 | 80.6 |
| Mass Balance | | 96 |

Use of borate in the load gave a higher overall recovery of transferrin in the eluate (80.6% recovery in the presence of borate, 53.6% recovery in the absence of borate). Also, less transferrin was present in the flow through (FT) when the load included borate (11.4% in the presence of borate, 31.3% in the absence of borate).

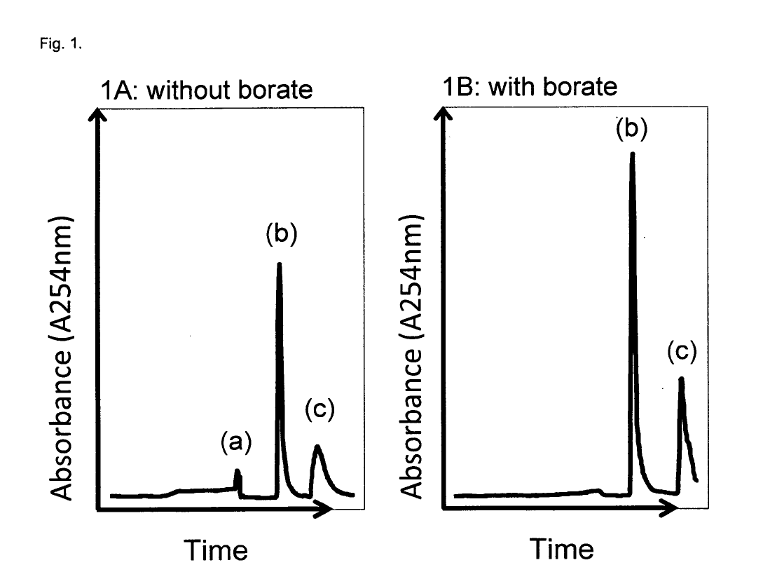

The data are also shown in FIG. 1. FIG. 1 shows that inclusion of borate in the load improves the binding of transferrin to the column. FIG. 1A is a trace for a Capto™ Q column for which tetraborate was not added to the transferrin prior to loading onto the column. FIG. 1B is a trace for a Capto™ Q column for which 824 mol.mol⁻¹ tetraborate was to the transferrin prior to loading onto the column.

Peak (a) relates to transferrin present in the wash following washing of the column. Peak (b) relates to transferrin present in the eluate. Peak (c) relates to transferrin recovered from the column following washing with NaOH.

The absence of a peak (a) on FIG. 1B shows that inclusion of tetraborate in the load improves the binding of transferrin to the column. The presence of peak (a) on FIG. 1A shows that loading transferrin on to the column in the absence of tetraborate results in some transferrin being undesirably eluted from the column during the washing step.

### Example 4

### Low Concentration Wash of Anionic Exchange Matrix

Capto™ Q chromatography was performed using a 0.66×15 cm (5.13 mL) column equilibrated in 15.7 mM potassium tetraborate. SP-FF eluate, from Example 1 (Table 4, wash regime C), was used as load material. The load was conditioned for Capto™ Q chromatography by the addition 27% (w/v) NaOH to achieve a load pH of 9.0-9.2 and water to achieve a conductivity ˜2.6 mS.cm⁻¹. Finally, solid potassium tetraborate was added to achieve a final concentration of 5 mM tetraborate (725 mol.mol⁻¹) increasing the load conductivity to ˜3.4 mS.cm⁻¹. A column capacity of 30 mg.mL⁻¹ matrix was used. Following load, the column was washed with 5 mM potassium tetraborate (10 CV), 15.7 mM potassium tetraborate (11 CV) and eluted with 150 mM potassium tetraborate pH 9.4 prior to regeneration of the column with 1 M NaCl, 0.5 M NaOH and 1 hour sanitisation with 1 M NaOH.

TABLE 7
Recovery of transferrin over Capto ™Q chromatography using
SP-FF eluate conditioned with 5 mM tetraborate in the load

| Fraction | Buffer | Recovery (%) |
|---|---|---|
| Load | pH 9.0, 3.42 mS · cm⁻¹ | 100 |
| | 5 mM potassium tetraborate | |
| FT | — | 1.9 |
| Wash 1 | 5 mM potassium tetraborate | 0.1 |
| Wash 2 | 15.7 mM potassium tetraborate | 0.2 |
| Elution | 150 mM potassium tetraborate | 93.2 |
| Clean 1 | 1M NaCl + 0.5% (w/v) Polysorbate 80 | 5.5 |
| Mass Balance | | 101.2 |

The data (Table 7) show that the loading of transferrin in the presence of tetraborate, followed by and low concentration wash and a high concentration wash results in low levels of loss of transferrin during each wash step and subsequently a very high level of transferrin (93.2% recovery) in the eluate.

### Example 5

### Mathematical Modelling of Low pH and Salt Concentration with Regards Removal of Yeast Antigen (HCP) and Recovery

The use of a low pH, high salt concentration wash regime to remove yeast antigens (YA) (i.e. host cell proteins (HCP)) and maintain eluate recovery on SP-FF was investigated further. A Design of Experiments (DoE) approach was employed to allow construction of a mathematical model of the effects of the two factors, pH and salt concentration, on the two responses, eluate recovery and YA clearance. A number of pH and salt concentrations in the range pH 3.66 to 5.34 and 0 mM to 6000 mM sodium chloride were studied. Load material was prepared by adjusting the pH and conductivity of culture supernatant to 5.11 and 3.57 mS.cm⁻¹ using glacial acetic acid and water. Fifteen Atoll 5 mm dia.×2.5 mm bed height MediaScout® MiniColumns (Atoll GmbH, Weingarten, Germany) containing 50 μL SP-FF matrix (GE Healthcare) were equilibrated with 50 mM sodium acetate pH 5.1. This load material was applied at 50 μL.min⁻¹ to achieve a 40 mg.mL⁻¹ matrix loading. Each column was washed first with the appropriate low pH sodium acetate buffer without NaCl (wash 1) and then with a second sodium acetate buffer of equivalent pH but containing the appropriate concentration of NaCl (wash 2) (Table 8). Each column was then eluted with 50 mM sodium phosphate, 150 mM sodium chloride pH 6.0 and cleaned with 1M NaCl, 0.5% (w/w) polysorbate 80. All chromatographic fractions were collected and the transferrin concentration in each fraction was estimated by reverse phase high performance liquid chromatography (RP.HPLC). YA levels were estimated by enzyme linked immunosorbent assay (ELISA). Recoveries and YA clearance were tabulated and are shown in Table 8.

TABLE 8
The effect of varying combinations of low pH and high
salt concentration in wash 2 on transferrin recovery
and YA clearance in the SP-FF eluate fraction.

| | Factors | | Responses | |
|---|---|---|---|---|
| Run | | NaCl | | YA |
| number | pH | Concentration (mM) | Recovery (%) | clearance (fold) |
| 1 | 4.00 | 5000 | 73.6 | 269 |
| 2 | 4.50 | 2525 | 76.2 | 324 |
| 3 | 3.66 | 2525 | 60.6 | 276 |
| 4 | 4.00 | 50 | 69.7 | 256 |
| 5 | 4.00 | 5000 | 68.0 | 286 |
| 6 | 4.50 | 2525 | 75.6 | 314 |
| 7 | 4.50 | 0 | 81.9 | 298 |
| 8 | 5.34 | 2525 | 26.5 | 213 |
| 9 | 5.00 | 5000 | 54.9 | 292 |
| 10 | 4.50 | 2525 | 73.6 | 345 |
| 11 | 4.50 | 6000 | 74.4 | 364 |
| 12 | 5.00 | 5000 | 21.9 | 208 |
| 13 | 5.00 | 50 | 81.8 | 356 |
| 14 | 4.00 | 50 | 56.1 | 287 |
| 15 | 5.00 | 50 | 80.8 | 383 |

Data for the recovery and YA clearance at each of the 15 wash conditions ('runs') were entered into the DoE software (Design Expert 6.0.10, Stat-Ease, Inc., Minneapolis, Minn., USA) to create a mathematical model of the design space. A computer uses ‘design points’ (i.e. experimental data points) to generate the mathematical model of the design space. The model was fitted for each of the responses as follows.

### (i) Recovery:

No transformation was required and a quadratic fit was used.
ANOVA (analysis of variance) data indicated that the fit was significant but that the salt² term was not required.
Therefore the salt² term was removed from the model and the data refitted to produce the following equation:

Recovery=−940.50069+(441.61018×p)+(0.043309×s)−(47.57735×p²)−(0.010263×p×s)

Where: ‘p’=pH and ‘s’=salt concentration in mM and recovery is % recovery.

### (ii) HCP(YA) Clearance:

No transformation was required and a quadratic fit was used.
ANOVA data indicated that a quadratic fit was not significant; therefore all terms were included for a cubic fit.
ANOVA data indicated that the fit was now significant but that the salt³ term was not required.
Therefore the salt³ term was removed from the model and the data refitted.
ANOVA data indicated that the fit was significant but that the salt² term was not required.
Therefore the salt² term was removed from the model and the data refitted.
ANOVA data indicated that the fit was significant but that the pH×salt² term was not required as it was aliased with the pH and pH³ terms.
Therefore the pH×salt² term was removed and the data refitted to produce the following equation:

HCP(YA)clearance=16388.65744−(10512.30681×p)−(1.70101×s)+(2257.17601×p²)+(0.78628×p×s)−(159.06573×p³)−(0.090182×p²×s)

Where: ‘p’=pH and ‘s’=salt concentration in mM and clearance is ‘fold-clearance’.

Using limits of greater than or equal to 60% and greater than or equal to 275-fold (‘≧’ means ‘greater than or equal to’), for acceptable performance of recovery and HCP(YA) clearance respectively, a graphical representation (overlay plot) of a design space was plotted (FIG. 2). The areas shaded in grey (i.e. I, III and IV) represent those combinations of pH and salt concentration where either recovery and/or HCP (YA) clearance fall outside the acceptable performance limits; whilst the area shown in white (i.e. II) represents those combinations where both pH and salt concentration fall within the acceptable performance limits (i.e. greater than or equal to 60% recovery and greater than or equal to 275-fold HCP (YA) clearance).

### Example 6

### Addition of Potassium Tetraborate to Capto Q Load

Addition of potassium tetraborate to the Capto Q load material in the range 1 to 1000 mol.mol was investigated. Load material was prepared by adjusting the pH and conductivity of SP-FF eluate (from Example 1) to 9.3 and 3.53 mS.cm⁻¹ using 0.5M NaOH and water. Sufficient 0.5M potassium tetraborate was added to 30 mL of this solution to achieve a load sample with a molar ratio of 1000 mol borate:1 mol transferrin. Individual load samples were prepared by serially diluting the 1000 mol.mol solution to 500, 100, 50, 10, 5, 2.5, and 1 mol.mol with the pH-adjusted, diluted solution prepared above. The conductivity of each load sample was measured and readjusted to 3.5 mS.cm⁻¹ with water. Eight Atoll 5 mm dia.×2.5 mm bed height MediaScout® MiniColumns containing 50 μL Capto Q matrix (GE Healthcare) were equilibrated first with 110 mM potassium tetraborate and then with 30 mM potassium tetraborate. A sufficient volume of each load sample was applied at 50 μLmin⁻¹ to achieve a 40 mg.mL⁻¹ matrix loading. Each column was washed first with 5 mM potassium tetraborate (wash 1) and then with 30 mM potassium tetraborate (wash 2) before being eluted with 110 mM potassium tetraborate and cleaned with 1M NaCl, 0.5% (w/w) polysorbate 80. All chromatographic fractions were collected and the transferrin concentration estimated by reverse phase high performance liquid chromatography (RP.HPLC). Recoveries were tabulated and are shown in Table 9.

TABLE 9
The effect of potassium tetraborate in Capto Q load
material between 1 and 1000 mol.mol on transferrin
recovery in the flow through and eluate fractions.

| | Recovery (%) | | | | | |
|---|---|---|---|---|---|---|
| | Flow | Wash | Wash | | Salt | Mass |
| Load conditions | through | 1 | 2 | Eluate | clean | Balance |
| 1000 mol.mol | 0.8 | 0.1 | 1.7 | 93.7 | 5.9 | 102.2 |
| tetraborate | | | | | | |
| 500 mol.mol | 2.2 | 0.2 | 2.5 | 91.0 | 5.9 | 101.8 |
| tetraborate | | | | | | |
| 100 mol.mol | 3.4 | 0.4 | 3.1 | 78.7 | 5.6 | 91.2 |
| tetraborate | | | | | | |
| 50 mol.mol | 6.0 | 0.5 | 2.6 | 75.5 | 4.8 | 89.4 |
| tetraborate | | | | | | |
| 10 mol.mol | 17.0 | 0.9 | 1.9 | 62.2 | 4.9 | 87.0 |
| tetraborate | | | | | | |
| 5 mol.mol | 12.2 | 1.1 | 1.1 | 48.6 | 4.0 | 87.0 |
| tetraborate | | | | | | |
| 2.5 mol.mol | 18.5 | 1.1 | 1.7 | 60.5 | 4.4 | 86.2 |
| tetraborate | | | | | | |
| 1 mol.mol | 14.0 | 1.0 | 2.3 | 65.4 | 4.6 | 87.3 |
| tetraborate | | | | | | |

The data indicated that between 50 and 1000 mol.mol the addition of potassium tetraborate to the load was having the desired beneficial effect and reducing the amount of trans-ferrin lost in the flow through fraction, resulting in an increase in the amount recovered in the eluate. Between 10 and 50 mol.mol there was a significant increase in the amount of trans-ferrin lost in the flow through resulting in a reduction in the amount recovered in the eluate. In order to better define the cut-off point for the effect of potassium tetraborate addition a second experiment was performed, essentially identical to that described above, but loading at 10, 20, 30, 40 and 50 mol.mol. The results of the second experiment are shown in Table 10.

TABLE 10
The effect of potassium tetraborate in Capto Q load
material between 10 and 50 mol.mol on transferrin
recovery in the flow through and eluate fractions.

| | Recovery (%) | | | | | |
|---|---|---|---|---|---|---|
| | Flow | Wash | Wash | | Salt | Mass |
| Load conditions | through | 1 | 2 | Eluate | clean | Balance |
| 50 mol.mol | 0.5 | 0.1 | 1.2 | 91.1 | 7.9 | 100.7 |
| tetraborate | | | | | | |
| 40 mol.mol | 7.3 | 0.4 | 3.6 | 78.8 | 5.7 | 95.8 |
| tetraborate | | | | | | |
| 30 mol.mol | 6.9 | 0.5 | 3.1 | 78.7 | 5.9 | 95.0 |
| tetraborate | | | | | | |
| 20 mol.mol | 7.5 | 0.5 | 3.4 | 91.0 | 5.8 | 108.2 |
| tetraborate | | | | | | |
| 10 mol.mol | 18.8 | 0.9 | 2.1 | 65.7 | 7.2 | 94.7 |
| tetraborate | | | | | | |

This second set of data indicated that between 20 and 50 mol.mol the addition of potassium tetraborate was having the desired beneficial effect and reducing the amount of transferrin lost in the flow through fraction, resulting in an increase in the amount recovered in the eluate. Below 10 mol.mol there was a significant increase in the amount of transferrin lost in the flow through resulting in a reduction in the amount recovered in the eluate.

In conclusion the addition of potassium tetraborate to the Capto Q load in the range 20 to 1000 mol.mol has a beneficial effect by reducing transferrin losses in the flow through with a resultant increase in the amount of transferrin recovered in the eluate.

### Example 7

### Addition of Potassium Tetraborate to Capto Q Wash 1

Addition of potassium tetraborate to the Capto Q wash 1 in the range 0 to 30 mM was investigated. Load material was prepared by adjusting the pH and conductivity of SP-FF eluate (from Example 1) to 9.3 and 3.53 mS.cm⁻¹ using 0.5M NaOH and water. Sufficient 0.5M potassium tetraborate was added to 50 mL of this solution to achieve a load sample with a molar ratio of 270 mol borate:1 mol transferrin. The conductivity of the load sample was measured and readjusted to 3.5 mS.cm⁻¹ with water. Nine Atoll 5 mm dia.×2.5 mm bed height MediaScout® MiniColumns containing 50 μL Capto Q matrix (GE Healthcare) were equilibrated first with 110 mM potassium tetraborate then with 30 mM potassium tetraborate. A sufficient volume of each load sample was applied at 50 μL.min⁻¹ to achieve a 40 mg.mL⁻¹ matrix loading. Each column was washed first with potassium tetraborate at either 0, 0.1, 0.5, 1, 2, 5, 10, 20 or 30 mM (wash 1) and then with 30 mM potassium tetraborate (wash 2) before being eluted with 110 mM potassium tetraborate and cleaned with 1M NaCl, 0.5% (w/w) polysorbate 80. All chromatographic fractions were collected and the transferrin concentration estimated by RP.HPLC. Recoveries were tabulated and are shown in Table 11.

TABLE 11
The effect of potassium tetraborate in Capto Q wash 1
between 0 and 30 mM on transferrin recovery
in the wash 1 and wash 2 fractions.

| | Recovery (%) | | | | | |
|---|---|---|---|---|---|---|
| Wash 1 | Flow | Wash | Wash | | Salt | Mass |
| conditions | through | 1 | 2 | Eluate | clean | Balance |
| 30 mM | 4.5 | 9.5 | 1.2 | 76.1 | 5.3 | 96.6 |
| tetraborate | | | | | | |
| 20 mM | 4.0 | 3.1 | 0.9 | 79.5 | 7.4 | 94.9 |
| tetraborate | | | | | | |
| 10 mM | 2.6 | 0.3 | 1.5 | 83.8 | 7.2 | 95.4 |
| tetraborate | | | | | | |
| 5 mM | 3.4 | 0.1 | 2.2 | 79.5 | 10.9 | 96.2 |
| tetraborate | | | | | | |
| 2 mM | 5.6 | 0.4 | 2.7 | 80.4 | 6.2 | 95.3 |
| tetraborate | | | | | | |
| 1 mM | 5.6 | 0.5 | 3.5 | 81.0 | 5.1 | 95.7 |
| tetraborate | | | | | | |
| 0.5 mM | 6.5 | 0.7 | 4.2 | 80.3 | 5.0 | 96.7 |
| tetraborate | | | | | | |
| 0.1 mM | 6.4 | 0.4 | 6.2 | 87.5 | 5.9 | 106.4 |
| tetraborate | | | | | | |
| 0 mM | 5.1 | 0.1 | 7.2 | 75.9 | 7.6 | 96.0 |
| tetraborate | | | | | | |

The data indicated that a concentration of between 0.5 and 20 mM potassium tetraborate in wash 1 was having the desired beneficial effect i.e. reducing the amount of transferrin lost in wash 2. Above 20 mM there was a significant increase in the amount of transferrin lost in wash 1 whilst between 0 and 0.1 mM there was an unacceptable increase in the amount of transferrin lost in wash 2.

### Example 8

### Addition of Sodium Tetraborate to Capto Q Load

Addition of sodium tetraborate to the Capto Q load material was investigated. Load material was prepared by adjusting the pH and conductivity of SP-FF eluate to 9.4 and 3.66 mS.cm⁻¹ using 0.5M NaOH and water. Sufficient 110 mM sodium tetraborate was added to half of this solution to achieve a load sample with a molar ratio of 270 mol borate:1 mol transferrin. The conductivity of the load sample was measured and readjusted to 3.61 mS.cm⁻¹ with water. Two Atoll 5 mm dia.×2.5 mm bed height MediaScout® MiniColumns containing 50 μL Capto Q matrix (GE Healthcare) were equilibrated first with 110 mM sodium tetraborate then with 30 mM sodium tetraborate. Sufficient of the 270 mol.mol load sample and a control of pH adjusted, diluted material containing no sodium tetraborate were applied at 50 μL.min⁻¹ to achieve a 40 mg.mL⁻¹ matrix loading. Each column was washed first with 5 mM sodium tetraborate (wash 1) and then with 30 mM sodium tetraborate (wash 2) before being eluted with 110 mM sodium tetraborate and cleaned with 1M NaCl, 0.5% (w/w) polysorbate 80. All chromatographic fractions were collected and the transferrin concentration estimated by reverse phase high performance liquid chromatography (RP.HPLC). Recoveries were tabulated and are shown in Table 12.

TABLE 12
The effect of sodium tetraborate in Capto Q load at
270 mol.mol on transferrin recovery in
the flow through and eluate fractions.

| | Recovery (%) | | | | | |
|---|---|---|---|---|---|---|
| | Flow | Wash | Wash | | Salt | Mass |
| Load conditions | through | 1 | 2 | Eluate | clean | Balance |
| 0 mol.mol | 16.9 | 0.9 | 3.2 | 63.2 | 5.2 | 89.5 |
| tetraborate | | | | | | |
| 270 mol.mol | 5.1 | 0.2 | 2.7 | 75.2 | 4.9 | 88.1 |
| tetraborate | | | | | | |

The data indicated that the addition of sodium tetraborate to the load at 270 mol.mol was having the desired beneficial effect i.e. reducing the amount of transferrin lost in the flow through fraction, resulting in an increase in the amount recovered in the eluate.

1. A process for purifying a transferrin solution, the process comprising:
a) adding tetraborate to a relatively impure solution of transferrin to provide a solution of transferrin and tetraborate;
b) applying the solution of transferrin and tetraborate to an anionic chromatographic material for which the transferrin has no specific binding activity such that the transferrin binds to the material;
c) washing the chromatographic material to which the transferrin is bound;
d) optionally, further washing the chromatographic material to which the transferrin is bound; and
e) optionally, recovering the transferrin from the anionic chromatographic material.

2. A process according to claim 1 wherein: in step (a), the solution of transferrin and tetraborate is at a ratio of at least about 20 mole tetraborate to about 1 mole transferrin.

3. A process according to claim 1 in which the washing of step (c) is carried out using a washing solution having a lower tetraborate concentration than the washing solution used for the washing of step (d).

4. A process according to claim 1 in which the washing of step (c) is carried out using a tetraborate solution of from about 0.1 mM to about 20 mM.

5. A process according to claim 1 in which the washing of step (d) is carried out using a tetraborate solution of from about 5 mM to about 60 mM.

6. A process according to claim 1 comprising:
a) adding tetraborate to a relatively impure solution of transferrin to provide a solution of transferrin and tetraborate at a ratio of at least 20 mole tetraborate to 1 mole transferrin;
b) applying the solution of transferrin and tetraborate to an anionic chromatographic material for which the transferrin has no specific binding affinity such that the transferrin binds to the material;
c) washing the chromatographic material with a tetraborate solution of from 0.1 mM to about 20 mM;
d) subsequently washing the chromatographic material with a tetraborate solution of at least 30 mM; and
e) optionally, recovering the transferrin from the anionic chromatographic material.

7. A process for purifying a transferrin solution, the process comprising an anionic chromatographic step and a cationic chromatographic step, in which:
the cationic chromatographic step comprises:
a) applying a relatively impure solution of a transferrin to a cationic chromatographic material for which the transferrin has no specific affinity such that the transferrin binds to the material;
b) washing the cationic chromatographic material with a wash buffer having a pH of from about 4 to about 5 and being substantially free of salts other than the salt which provides the buffering effect;
c) subsequently washing the cationic chromatographic material with a wash buffer having a pH from about 4 to about 5 and a salt concentration of from about 50 mM to about 5 M; and
d) recovering the transferrin from the cationic chromatographic material; and
the anionic chromatographic step comprises:
a) adding tetraborate to transferrin obtained from step (d) of the cationic chromatographic step to provide a solution of transferrin and tetraborate;
b) applying the solution of transferrin and tetraborate to an anionic chromatographic material for which the transferrin has no specific activity such that the transferrin binds to the material;
c) washing the chromatographic material to which the transferrin is bound;
d) optionally further washing the chromatographic material to which the transferrin is bound; and
e) optionally, recovering the transferrin from the anionic chromatographic material.

8. A process according to claim 1 wherein the tetraborate solution used in the washing of step (c) of the anionic chromatographic step is from about 2.5 mM to about 7.5 mM.

9. A process according to claim 8 wherein the tetraborate solution used in the washing of step (c) of the anionic chromatographic step is about 5 mM.

10. A process according to claim 1 wherein the tetraborate solution used in the washing of step (d) of the anionic chromatographic step is from about 20 mM to about 40 mM.

11. A process according to claim 10 wherein the tetraborate solution used in the washing of step (d) of the anionic chromatographic step is about 30 mM.

12. A process according to claim 1 wherein the tetraborate is potassium tetraborate or sodium tetraborate.

13. A process according to claim 1 wherein the anionic chromatographic material comprises a functional ligand selected from: a diethylaminoethyl (DEAE) group and a quaternary amine group.

14. A process for purifying a transferrin solution, the process comprising a cationic chromatographic step comprising the steps of:
a) applying a relatively impure solution of a transferrin to a cationic chromatographic material for which the transferrin has no specific affinity such that the transferrin binds to the material;
b) washing the chromatographic material with a wash buffer having a pH of from about 4 to about 5 and having a salt concentration of less than 200 mM;
c) subsequently washing the chromatographic material with a wash buffer which:
i) which lies within a design space defined by both of the following equations:
Recovery=−940.50069+(441.61018×p)+(0.043309×s)−(47.57735×p)−(0.010263×p×s) (I)
Host cell protein clearance=16388.65744−(10512.30681×p)−(1.70101×s)+(2257.17601×p²)+(0.78628×p×s)−(159.06573×p³)−(0.090182×p²×s) (II)
where p=pH, s=salt concentration in mM, Recovery is greater than or equal to 60% and YA clearance is greater than or equal to 275-fold; and
ii) has a pH of from 4 to 5 and a salt concentration of from 50 to 5000 mM; and
iii) has a higher salt concentration that the wash buffer of step (b); and
d) optionally, recovering the transferrin from the cationic chromatographic material.

15. A process according to claim 7 in which the pH of the wash buffer of step (b) of the cationic chromatographic step is from about 4.3 to about 4.7.

16. A process according to claim 15 in which the pH of the wash buffer of step (b) is about 4.5.

17. A process according to claim 7 in which the wash buffer of step (b) of the cationic chromatographic step has a salt concentration of less than 50 mM.

18. A process according to claim 17 in which the wash buffer of step (b) does not comprise salt other than the salt which provides the buffering effect.

19. A process according to claim 7 in which the wash of step (c) of the cationic chromatographic step:
(i) lies within a design space defined by both of the following equations:
Recovery=−940.50069+(441.61018×p)+(0.043309×s)−(47.57735×p2)−(0.010263×p×s) (I)
Host cell protein clearance=16388.65744−(10512.30681×p)−(1.70101×s)+(2257.17601×p2)+(0.78628×p×s)−(159.06573×p3)−(0.090182×p2×s) (II)
where p=pH, s=salt concentration in mM, Recovery is greater than or equal to 60% and YA clearance is greater than or equal to 275-fold; and
(ii) has a pH of from 4 to 5 and a salt concentration of from 50 to 5000 mM.

20. A process according to claim 7 in which the pH of the wash buffer of step (c) of the cationic chromatographic step is from about 4.3 to about 4.7.

21. A process according to claim 20 in which the pH of the wash buffer of step (c) is about 4.5.

22-27. (canceled)
